(19) United States
(12) Patent Application Publication
(10) Pub. No.: US 2005/0266020 A1
(43) Pub. Date: Dec. 1, 2005

(54) Modular transport systems for molecular substances and production and use thereof

(75) Inventors: Gerald Boehm, Halle (DE); Rainer Rudolph, Halle (DE); Ulrich Schmidt, West Leederville (AU); Dirk Esser, Cambridge (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ACGT ProGenomics AG, Halle (DE)

(21) Appl. No.: 11/120,543

(22) Filed: May 2, 2005

Related U.S. Application Data
(63) Continuation of application No. 10/129,428, filed on Nov. 8, 2002, now abandoned.

(30) Foreign Application Priority Data
Nov. 3, 1999 (DE) 19952957.4

(51) Int. Cl. A61K 49/00; A61K 39/12; C12N 7/00; A61K 48/00

(52) U.S. Cl. 424/199.1; 435/235.1; 424/9.6; 424/93.2

(57) ABSTRACT

The invention relates to transport systems for molecular substances, comprising a mosaic of recombinant partial units (individual components). The invention further relates to production of the molecular transport system and use thereof.

This invention involves transport systems for molecular substances, with the transport systems being made in a mosaic-like fashion from partial units produced separately and recombinantly (single building blocks), as well as procedures for producing modular transport systems and their use.

### FIELD OF INVENTION AND STATE OF TECHNOLOGY

Medical gene therapy enables a permanent and gentle therapy for a series of serious diseases, and represents, according to the general opinion, an important alternative to traditional medical methods like for example chemotherapy. The general procedure is based on the targeted insertion of therapeutically effective material, mostly based on nucleic acid, into somatic cells. The aim of gene-therapeutic treatments is either a therapy of congenital genetic defects (classical gene therapy), a therapy of diseases acquired by infection (for example EBV infection, HIV infection), or a tumour therapy. Under this premise, the different concepts for treating serious diseases are summed up as gene-therapeutical treatments.

The classical gene therapy deals with (inherited) genetic defects and the associated diseases, which can be put down to a mostly unique cause (normally a dysfunctional protein). Some of these monocausal diseases are for example ADA deficiency, hemophilia, Duchenne muscular dystrophy, and cystic fibrosis, for which gene-therapeutic methods have been tested since around 1990 for therapy. The aim is the replacement or the complementation of a missing protein after specific insertion of suitable genetic material into the body cell. In contrast to this, the infectiological gene therapy attempts the therapy of viral or bacterial infections by elimination of the relevant pathogen; the cells affected by viruses shall normally be treated or devitalized before new infectious viruses maturate. Main target direction of present research efforts is the HIV infection. In contrast to this, the gene therapy of tumour diseases intends the transport of toxic substances into neoplastic cells, or the application of analogous principles (apoptosis, immune stimulation) for selective elimination of malignant cells.

Basically, in gene therapy two methodically different approaches according to the state of the technology are discussed: (i) Isolated cells are transformed extracorporally (in vitro) with the genetic material, often by cell-type unspecific retroviruses; afterwards, the transformed cells are reimplanted into the donor body. (ii) The target cells are infected in vivo with specific vectors; here, especially replication-deficient retroviruses or adenoviruses or adeno-associated viruses are used. But there are also physical systems used like condensated DNA, virus-like particles and others.

The inserted genetic material, mainly DNA, may either integrate into the chromosome (permanent expression, for example for the therapy of congenital, monocausal diseases) or be expressed transiently; this is sufficient for example for an infectiological therapy or a tumour therapy. In these cases, it is also possible to insert antisense RNA or ribozymes instead of DNA, or therapeutic agents like peptides or proteins are used.

Despite successful experimental beginnings for gene therapy, there are, however, also some problems known according to the current state of the technology. Replication-competent retroviruses as vectors, for example, may lead to serious diseases in animal models (W. F. Anderson, Hum. Gene Ther. 4, 1-2, 1993; Otto, Jones-Trower, Vanin, Stambaugh, Mueller, Andersen & McGarrity, Hum. Gene Ther. 5, 567-575, 1994). There is often only little efficiency of cell transformation with in vivo methods, and the specificity of the cellular targeting using retroviruses as vectors is usually not given. The systems are lavish regarding the production according to GMP conditions; the production of viral vectors with the help of packaging cell lines, in turn, results in a lavish analysis of the preparations. These and other disadvantages to the state of the technology, described in the following, shall be circumvented according to the invention by new modular vector systems as transport vehicles for molecular substances.

Almost all well-known viruses and phages have a capsid that is build up of at least one or several proteins and in which the viral genome is encapsidated. The capsids show a defined morphology, which is characteristic for a certain virus or a phage. Icosahedral or filamentous capsids are built particularly often. Table 1 shows an overview concerning the morphology of well-known viruses. There are numerous examples that those capsids can be built up in vitro from isolated viral proteins without the genome of the virus or cellular factors being present. The structures resulting from that, consisting of empty or filled protein coats, are described as virus-like or virus-analogous particles.

TABLE 1
Morphology of well-known viruses and virus families

| Morphology | Representatives (viruses or phages) |
|---|---|
| Amorphous or | Umbravirus; Tenuivirus |
| unknown | |
| bacilliform | Baculoviridae; Badnavirus; Barnaviridae; |
| | Filoviridae; Rhabdoviridae |
| filamentous | Capillovirus; Carlavirus; Closterovirus; |
| | Furovirus; Inoviridae; Lipothrixviridae; |
| | Potexvirus; Potyviridae; Tobamovirus; |
| | Tobravirus; Polydnaviridae |
| helical | Hordeivirus; Paramyxoviridae; Trichovirus |
| icosahedral | Adenoviridae; Astroviridae; Birnaviridae; |
| | Bromoviridae; Caliciviridae; Caulimovirus; |
| | Circoviridae; Comoviridae; Corticoviridae; |
| | Dianthovirus; Enamovirus; Hepadnaviridae; |
| | Herpesviridae; Idaeovirus; Iridoviridae; |
| | Lviviridae; Luteovirus; Machlomovirus; |
| | Marafivirus; Microviridae; Necrovirus; |
| | Nodaviridae; Papovaviridae; Partitiviridae; |
| | Parvoviridae; Phycodnaviridae; |
| | Picornaviridae; Reoviridae; Rhizidiovirus; |
| | Sequiviridae; Sobemovirus; Tectiviridae; |
| | Tetraviridae; Tombusviridae; Totiviridae; |
| | Tymovirus |
| isometric | Cystoviridae; Geminiviridae |
| oval | Poxviridae |
| pleomorphic | Coronaviridae; Hypoviridae; Plasmaviridae |
| spheric | Arenaviridae; Arterivirus; Bunyaviridae; |
| | Flaviviridae; Orthomyxoviridae; |
| | Retroviridae; Togaviridae |
| lemon-shaped | Fuselloviridae |
| unclassified hyper- | Bacilloviridae; Guttaviridae |
| thermophilic phages | |
| and viruses | |
| phages with caudal | Myoviridae; Podoviridae; Siphoviridae |
| appendage | |

When using such virus-like particles as transport vehicles, the coat proteins used have to be produced in a suitable expression system. Especially eukaryotic systems are used like for example baculovirus-infected insect cells; or prokaryotic systems like for example recombinant E. coli. With eukaryotic expression systems, complete virus-like particles are built within the cells; furthermore, it is possible to produce virus envelopes that are built from different viral proteins, for example polyomavirus coat proteins VP1 and VP2 (An, Gillock, Sweat, Reeves & Consigli, J. Gen. Virol. 80, 1009-1016, 1999). The disadvantage of these methods is above all the lavish, cost-intensive production of the viral capsids. During the expression of viral coat proteins in E. coli, complete viral capsids are not built in the cells but instead capsomeres are produced which have to be isolated from the cells and assembled in vitro into virus-like particles. There is, however, the possibility to gain great amounts of the coat proteins, yet a suitable protocol for in vitro assembly of the respective coat protein has to be found. Furthermore, with this method there is the possibility to build up viral capsids of different viral coat proteins, which occur naturally in the respective virus, for example herpes simplex viral capsids can be built of VP5, VP19C and VP23 (Newcomb, Homa, Thomsen, Trus, Cheng, Steven, Booy & Brown, J. Virol. 73, 4239-4250, 1999). However, the possibility to build up viral capsids from different, modified partial units, is not described in the state of the technology.

The VP1 protein of polyomavirus assembles in vitro under suitable solvent conditions spontaneously into a virus-like shell; this property of the wild-type protein is already known according to the state of technology. This process can be used to produce a molecular transport vehicle for the targeted transfer of molecules (for example for therapeutic agents), which are encapsidated in the coat of the virus-like particle. Apart from important structural investigations, the polyoma VP1 protein is extremely well examined regarding its molecular biological and pathological properties. The published work include production, purification and characterization as well as structure and assembly of the protein (Rayment, Baker & Caspar, Nature 295, 110-115, 1982; Garcea & Benjamin, Proc. Natl. Acad. Sci. U.S.A. 80, 3613-3617, 1983; Slilaty & Aposhian, Science 220, 725-727, 1983; Leavitt, Roberts & Garcea, J. Biol. Chem. 260, 12803-12809, 1985; Moreland, Montross & Garcea, J. Virol. 65, 1168-1176, 1991; Griffith, Griffith, Rayment, Murakami & Casper, Nature 355, 652-654, 1992). Especially the assembly in vitro is documented in detail according to the state of the technology (Slilaty, Berns & Aposhian, J. Biol. Chem. 257, 6571-6575, 1982; Salunke, Caspar & Garcea, Cell 604, 895-904, 1986; Garcea, Salunke & Caspar, Nature 329, 86-87, 1987; Salunke, Caspar & Garcea, Biophys. J. 56, 887-900, 1989). The potential use of vehicles, constructed in this way and consisting of subunits of the naturally occurring protein, is in principle described for gene transfer (Forstová, Krauzewicz, Sandig, Elliott, Palková, Strauss, & Griffin, Hum. Gene Therapy 6, 297-306, 1995). In WO 97/43431, a vehicle for the transport of molecular substances is described, comprising at least one capsomer derived from a virus and showing a modification on one of its sides, so that the molecular substance can be bound to the capsomer.

Until now, methods for in vitro assemblies of polyoma viral capsids in a mosaic-like fashion or other virus-analogous particles which are composed of various modified partial units or similar polymodified partial units have not been described as the state of the technology yet, since modifications of partial units often result in loss of the assembly competence of the partial units.

Therefore, it is the task of this invention to provide modular transport systems for molecular substances which are composed of differently modified partial units or similar polymodified partial units, and do not have the described disadvantages of the state of the technology.

In order to solve the task, transport systems for molecular substances are provided by the invention according to claim 1, containing recombinantly produced partial units based on amino acids, including:
at least two partial units modified differently to each other, and/or
one or several partial units modified differently twice, and
alternatively unmodified partial units,
   with the partial units being able to make a transport system like a mosaic and in addition molecular substances can be encapsidated into the transport system.

Advantageous forms of the transport systems as well as methods for production and use of the transport systems follow from the subclaims and from the description.

### DESCRIPTION

The use of natural viruses or virus-analogous systems for the transfer of nucleic acids into cells (gene therapy) is an important field of research in the area of molecular medicine. Here, a special challenge is the production of a vector system (transport system) that, according to the state of the technology, excludes or minimizes disadvantages concerning gene therapeutic treatments.

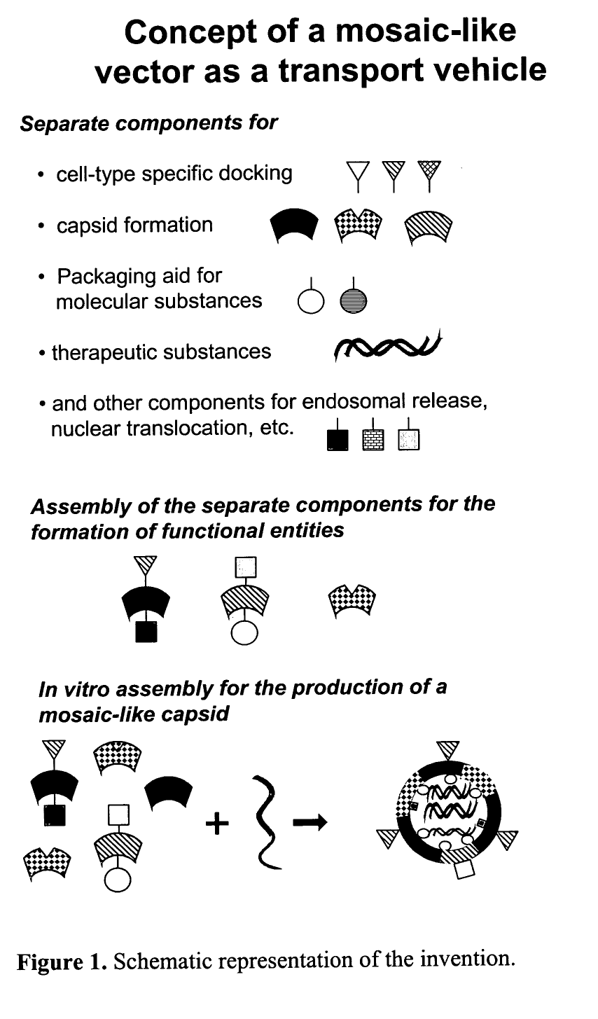

In this invention, a transport system for molecular substances is described which can be assembled in vitro from different single components. This is achieved by the use of molecular components or partial units (“modules”), which consist of proteins following this invention. These partial units can be modified in different ways by this invention, i.e. the amino acid sequences of the partial units can be changed, prolonged or shortened, in order to integrate desired properties from these modules into the transport system. The modules can particularly also contain functional domains from other proteins by fusions and insertions. The single functional modules can be composed in vitro (assembled), either directly due to their molecular properties, or, for example for the case that the modules are not assembly-competent, by coupling to special modules that show the required assembly competence. Within the limits of the invention, virus-like particles that have certain functions due to their composition can be built up in a mosaic-like fashion. A special advantage is the fact that the molecular composition of the transport systems can be determined stoichiometrically. The emerging virus-like particles can be used to transport molecular substances like nucleic acids, peptides or proteins efficiently and targeted into the interior of eukaryotic cells. A way of performance of the invention is presented schematically in FIG. 1.

From the invention, the transport systems can include modified partial units of the viruses and phages, shown in table 1, or of macromolecular protein assemblies with an internal cavity like proteasomes or chaperones and alternatively unmodified partial units of it. Following the invention, the transport systems can include monomers, dimers or oligomers of partial units. From the invention, those transport systems are preferred whose partial units are derived from the polyoma virus VP1 protein or modified partial units of it. Furthermore, those transport systems are preferred whose partial units are derived from phage proteins, especially of such phages that show hosts of thermophile or hyperthermophile origin and thus still form stable structures also at high environmental temperatures (≧70° C.). Here, the SSV1 particle (Fuseolloviridae) has to be emphasized, which infects the archaeobacteria Sulfolobus shibatae. This representative of the phages is hyperthermophile due to its host specificity, therefore stable also at high temperatures and can so be used optimally for a multitude of applications in the field of biotechnology and medicine. It is able to develop a very stable protein coat, and the building blocks can be produced recombinantly easily. Similar representatives of thermophile or hyperthermophile phages can also be found, for example, from the Lipothrixviridae (representatives: TTV1, TTV2, TTV3). The thermophile and hyperthermophile representatives of the Bacilloviridae (example: TTV4, SIRV) and Guttaviridae (example: SNDV), which can also be used in such processes, where amongst other things the stability of a protein coat (formed from the phage proteins) is relevant, are not further classified yet.

The modified partial units are rather produced recombinantly by the invention.

By the invention, the transport systems include at least two partial units modified differently from each other, in which “differently modified” means that the partial units show different modifications or the partial units show the same modification at different positions of the partial unit.

The transport systems from this invention can also include one or several partial units modified at least twice, and partial units modified differently twice are preferred.

From the invention the transport systems can in addition include unmodified partial units.

From the invention, the recombinantly produced partial units can be modified by point mutations or by insertion, removal or change of one or several amino acids, peptide or protein sequences or protein domains at the terminus/the termini and/or in the sequence of the partial unit.

The modifications can for example be labellings, so for example fluorescent dyes, polyethylene glycol, oligonucleotides, nucleic acids, peptides, peptide hormones, lipids, fatty acids or carbohydrates.

The partial units may also show modifications that cause an improved binding affinity of the partial units to molecular substances, for example proline-rich sequences, WW sequences, SH3 domains, biotin, avidin, streptavidin, or polyionic sequences. Such modifications are located preferrably at the inside of the transport system.

Furthermore, modifications are planned by the invention, by which an improved uptake into the desired target cells can be achieved, for example by carbohydrate structures, proteins or protein domains, antibodies or modified antibodies, antigens, or isolated receptor binding domains of ligands or other substances or sequences that can mediate a binding to receptors on the surface of the target cells.

Moreover, the partial units can show modifications by the invention, by which a transport in particular organelles of the target cells (for example nucleus, mitochondria, endoplasmatic reticulum) or a transport out of the target cells is possible. This modification that causes an improved uptake into target cells, organelles, or a transport out of the target cells, are mostly at the outside of the transport system or are a component of the molecular substance which has to be transported.

The procedure for producing the transport systems by the invention contains the following steps:
recombinant expression of the partial units,
release of the partial units by lysis of the hosts cells,
creating a contact of the partial units in the desired stoichiometric relations in order to compose (to assemble) the transport system like a mosaic, and
creating a contact with molecular substances, either before or during the assembly, in order to encapsidate the molecular substances into the transport system.

The starting point described by this invention is an advantageous alternative to the present customary methods of experimental gene therapy, e.g. the use of viruses, liposomes or physical systems. When using replication-deficient viruses, for example, extensive examinations are necessary to guarantee the biological and therapeutic safety of these vectors. In contrast to these systems, this invention describes a method that has a simple, gradual in vitro construction of a virus-analogous particle as a basis, consisting of parts composed in a mosaic, and is therefore very safe regarding a medical or therapeutic application.

The advantages of the modular construction of artificial viral vector systems described in this invention compared to traditional, mostly retroviral systems, are summarized in the following.
Safety problems of the vectors which are often discussed to occur on the construction of artificial replication-deficient viruses (retroviruses, adenoviruses) are avoided, as here not complex, potentially pathogenic viruses are reduced by some properties, but only artificial associates, for example built up from proteins, are extended with required properties. The complete synthesis of the capsids in vitro enables the implementation of maximum demands on a safe system for gene-therapeutic applications. The single components are completely uncoupled from the therapeutic starting point; the therapeutically effective substance (DNA, RNA, or analogous molecules) does not include any information about the production of the molecular vehicle. Thus, the occurrence of replication-competent species can be excluded completely. Furthermore, the final vectors do not include any genetic information about the construction plan of the particle, so disadvantageous potential danger by recombination events are completely out of question.
High purity and homogeneity of the systems are guaranteed by the in vitro assembly of components, which can be produced separately in high quality according to the state of the technology. Via highly specialized affinity purification steps (at the moment, for example, this is done by a self-splicing protein at an affinity matrix) of the isolated components; all unwanted, problematic components (contaminating DNA, bacterial proteins and endotoxins) can be removed efficiently.
The synthetically (recombinantly) produced virus capsids can be fluorescence-labelled with the help of a unique cysteine residue in each subunit of a particular variant or can be provided with molecular labelling, suitable for PET (positron emission tomography), and other methods for localization. These labellings enable the detection of the vectors within (non-fixed, i.e. living) cells by means of confocal fluorescence microscopy as well as—in a time-resolved manner—within complete, living organisms.
The fluorescence labelling of all components of the system allows the quality control regarding the composition of the preparations through FACS analyses of the capsids, in which the composition can be detected precisely by statistical counting of single particles.
In vitro as well as in vivo application of the vectors is possible in principle.

Advantages of modularly built up, artificial virus-analogous vector systems according to the invention over systems that are built up from a homogenous component (for example virus-like particles described in the literature) are summed up in the following.
An (uncoupling) of the minimal required single steps of gene therapeutic methods is possible: (i) the specific packaging of a disease-specific therapeutic agent, (ii) the cell-specific targeting (cell tropism), (iii) uptake and release from endosomes, (iv) compartment-specific translocation within the cell, and (v) selective initiation of action of the therapeutic substance.
The modular structure of the vehicle allows the selective integration of all necessary functions into the synthetic particle, with only these functions to be taken into consideration that are required for this kind of application. For example, the transport of a disease-specific therapeutic agent (for example DNA that allows the expression of the therapeutic gene by means of a tissue-specific promoter) does not necessarily require a domain for cell-type specific targeting.
The exactly dosable composition of the particle enables an integration of the various required functions in the dosage which is exactly necessary for it. A reduction or avoidance of unwanted side-effects at high therapeutic dosage is achieved by that.
Different therapeutic target directions do not require the working out of completely new systems or production methods, but only the introduction of single new building blocks or a modification of existing components of the complete system. In the scope of a tumour therapy, for example, an anti-tumour agent can be transported into the tumour tissue, with tumour cells of a particular type being specifically addressed by a corresponding receptor binding domain. A variation of the receptor-binding domain enables the transport of the same therapeutic into tumour cells of another type. Besides, therapeutic substances acting differently can be applied in an otherwise native system for example by variation of certain domains to the specific packaging of a therapeutic (for example DNA, RNA, peptides or proteins).
Potentially weak points of the therapeutic approach can easily be identified by comparative testing of different functional modules and be eliminated afterwards, and a better understanding for the basic molecular biological processes in natural viruses can be achieved.

The single building blocks (partial units) of the transport systems can be created, so that they have individual functional properties. The mosaic-like composition (assembly) is carried out in vitro and can be determined by stoichiometric additions of building blocks and suitable assembly conditions. The building blocks of the transport system are usually produced recombinantly. Therefore, the generated virus-like envelope structures (capsids) can show the desired properties and functions for the respective application. New functions and fields of applications can be provided and supplemented by the addition of further modules, with the single modules being produced independent of each other regarding their functional and molecular properties. Transport systems for molecular substances produced like this are especially suitable for applications in the field of gene therapy, also for the specific insertion of agents like, for example, DNA or proteins into eukaryotic cells.

According to an application form of this invention, the polyomavirus VP1 protein is changed in its natural properties and a transport system is provided with properties that are not described in the current state of the technology. The VP1 protein can be changed for example so that unwanted natural properties like the binding to a specific receptor on the surface of cells, for example kidney epithelial cells of the mouse, are eliminated without affecting the assembly. On the other hand, the inclusion mechanism into eukaryotic cells can be modulated by the introduction of specific new sequences; certain sequence motifs stimulate the uptake into cells.

The three-dimensional structure of the protein is well-known (Stehle, Yan, Benjamin & Harrison, Nature 369, 160-163, 1994). Within the scope of the invention it was possible to show that a functional module in the form of a domain, e.g. for the receptor-specific docking (cell-type specific targeting) can be inserted into at least two loop segments at the outside of the protein (amino acid positions 148 and 293) (cf. example 4).

Furthermore, it was possible to show that a modulation of the disulfide bridge pattern may occur by a change in the cysteine composition of the subunits as well as of the assembled capsids. In this way the biological stability of the particles can be varied.

According to the invention, the variants of the VP1 protein are produced with special, new properties that the naturally occurring wild-type protein does not show. Here, it has proved to be especially advantageous that a production and purification of the modified VP1 proteins can occur via a method described in example 1. Furthermore, changes can be undertaken by means of genetic engineering (point mutations, see example 2, 3, and 5) and additional (functional) domains, peptides or proteins can be fixed to the termini of the VP1 protein or implanted into the sequence of the VP1 protein (cf. example 4). These functional units can extend the properties of the coat protein, for example, by functions concerning the specific receptor-docking, the efficient uptake into the target cells, or the binding and packaging of the molecule which is to be transported. Especially the single functional units can be combined within an envelope by assembling the different coat proteins in a mosaic-like fashion, so that multi-functional virus-like particles are formed. Here, the optimal amount of each functional unit within a single virus capsid can be set according to the kind of application. An artificial, virus-analogous particle constructed like that can be used in many ways, but can be used especially for the specific transfer of therapeutically effective molecular substances into target cells.

This invention describes modular transport systems, built up in a mosaic-like fashion, for therapeutic substances, in which an easy and quick adaptation of the system to the respective application is enabled. An area of application of the invention can be the therapy of infectious diseases like for example AIDS. There, a multiplication of the HIV virus in CD⁴⁺ lymphocytes takes place, which leads to the described symptoms. The infected cells present the viral protein gp120 on their surface during the late phase of infection, which binds to the natural receptor CD4 and arranges the uptake of the virus into the cell. This mechanism can be used for the cell-specific targeting by modifying the surface of the transport system described in this invention, either with the receptor CD4 or with single CD4 domains, which are necessary for the binding to gp120. As the interaction of CD4 with gp120 is highly specific and does not occur in any other tissue of the body, such a transport vehicle only interacts with lymphocytes that have already been successfully infected by HIV, that is, the therapeutic substance is transported exclusively into infected cells as desired.

DNA can be used as a therapeutic substance which encodes intracellularly acting antibodies, that in turn bind specifically to HIV proteins and therefore neutralize them in their function. The therapeutic DNA may be inserted into the cell as single or double-stranded nucleic acid. In the case of double-stranded DNA, the inclusion into the particles can occur by inserting single, modified modules that interact with dsDNA. Such a module can carry basic sequences at the inside of the particle which interact with DNA. Moreover, a coupling of DNA-intercalating substances for binding double-stranded DNA is possible. Single-stranded DNA, in turn, can also be directed into the particles by using modules with ssDNA binding proteins. A coupling of sequence-specific oligonucleotides to the inside of the particle would be possible, which arrange a packaging with the therapeutic ssDNA by means of hybridization.

Another starting point for the HIV therapy would be the packaging of ribozymes that have a specific recognition sequence for HIV-RNA. The viral RNA is split catalytically and inactivated upon binding of the ribozymes. In this case, the packaging of ribozymes can be done by modules that have RNA binding domains or analogous building blocks for the encapsidation of ssDNA with oligonucleotide-modified vehicles. The therapy can also occur by inserted proteins or peptides as an alternative to nucleic acids. Inserted transdominant (modified) proteins can compete with native HIV proteins in the cell and so inhibit their function. Also, peptides or synthetically modified peptides can inhibit the effect of certain HIV proteins, for example of HIV protease. Furthermore, it is possible to direct the proteins inside the cell by corresponding signal sequences, for example into the nucleus with the help of a nuclear translocation sequence of the large T-antigen of the virus SV40. This is necessary for an interaction with factors localized in the nucleus, like for example the HIV-Tat protein, which among other things serves as transcriptional factor in the cell nucleus and drastically increases the transcription of viral proteins. Proteins can be included into the transport vehicles by binding to modules which contain sequence-specific binding domains in such a way that the bound proteins are brought into the inside of the vehicle. Besides, the mentioned proteins or peptides can be fused directly to the vehicle building blocks, in such a way that there is a recognition sequence for HIV protease or a cellular protease in between which releases the protein or peptide intracellularly and again specifically in infected cells.

Another application of this invention is the application of anti-tumour agents by malignant diseases. Therefore, the vehicles have to contain building blocks that guarantee the transport of the agent into tumour tissue. According to the type of tumour, this occurs, for example, by antibodies located on the surface of the particle, which bind to tumour antigens, which are exclusively or to a maximum extent only available on tumour cells. Solid tumours require a sufficient blood supply and therefore secrete growth factors that initiate the formation of new blood vessels in the tumour tissue. The epithelial cells of newly formed blood vessels express increased amounts of plasma membrane bound integrin receptors. These receptors specifically recognize the sequence RGD (arginine-glycine-aspartate) and induce a receptor-mediated endocytosis of ligands containing RGD. This property can also be used for targeting tumour cells and epithelial tissue connected to it, by integrating RGD exposing modules into the transport vehicle, so that an inclusion of the therapeutic substance into the tumour tissue occurs. A combination of different receptor-binding properties induces a therapy apart from an improved tissue specificity, which attacks the tumour on several sites and at the same time reduces the formation of drug resistant cells.

Nucleic acids like single- or double-stranded DNA or RNA can be used as agents. The proteins encoded by them can for example initiate apoptosis in the cell by interfering with the cellular signal transduction cascades at the corresponding sites. For an extended tumour specificity and therefore a higher safety, promoters can be used for transcription which are preferentially active in tumour cells. Peptides which induce an inhibition of matrix metalloproteinases can be used in the same way. Especially the inhibition of MMP-2 and MMP-9 by specific, short peptide sequences can here show an effective action.

Apart from the mentioned nucleic acids, also proteins and peptides can be packaged which initiate apoptosis or necrosis. Suitable for this are, for example, catalytic domains of bacterial toxins (for example diphtheria toxin, cholera toxin, botulinus toxin, and others), which inhibit the protein biosynthesis of the cell with high efficiency and thus trigger necrosis. Here, it can be an advantage that only few molecules are necessary to kill a cell. Another therapeutic starting point represents the transport of thymidine kinase of herpes simplex virus into tumour cells. This enzyme phosphorylates nucleotide building blocks and shows a reduced substrate specificity compared to the cellular kinases, so that artificial nucleotides like, for example, ganciclovir are also phosphorylated. Phosphorylated ganciclovir is built into newly synthesized DNA strands during DNA replication and leads to stop of replication, which in turn prevents the cell division.

Basically, the invention described here can also be applied for correcting inherited genetic defects like ADA deficiency, hemophilia, Duchenne atrophy, and cystic fibrosis. These diseases are monocausal, that is, they can be put down to a defect of one single gene. Therefore, the insertion of this gene in correct form is usually sufficient to compensate or reduce the symptoms. For this application, a stable gene expression has to be achieved, either by stable episomal vectors or by an integration of the therapeutic DNA into cellular chromosomes. Therefore, the transmitted nucleic acids can include sequences that make an integration easier. A single-stranded DNA, for example, can be used which carries ITR sequences (inverted terminal repeats) from Adeno-associated virus at its ends, which contribute to the chromosomal integration. Besides, proteins can be transported into the cell, apart from the therapeutic DNA or RNA, which catalyze an integration activity like for example HIV integrase, or Rep78 and Rep68 from Adeno-associated virus.

The expression of correcting genes can occur ideally under control of the natural promoters, by which an adopted regulation is guaranteed at the same time. In many cases, a cell type-specific targeting of the transport vehicle is therefore not necessary. For example, hemophilia patients can produce the missing factors from the blood coagulation cascade in muscular tissue, with the factors being fused with a suitable signal sequence, so that they are secreted from the cell and reach their place of action, the blood stream.

In all cases of a practical application, an efficient release of the therapeutic substances within the cell is necessary, that is, the substance has to pass through the endosomal membrane successfully. This function can be realized by hemolysines, especially thiol-activated cytolysines, translocation domains of bacterial toxins, or certain viral proteins like, for example, the adenovirus penton protein. These functions can be included into the transport vehicle which is composed in a mosaic as a part of the vector system described in this invention. Furthermore, this function can be taken over by chemical substances like, for example, polycations or dendrimers. The corresponding component either has to be brought to the surface of the particles or has to be encapsidated in the particles.

It may also be necessary for many applications to keep the immunogenicity of the transport system as well as of the therapeutic agent as little as possible. The humoral immunogenicity of the transport vehicles themselves and the recognition and elimination by macrophages can be achieved by the invention by a masking with polyethylene glycol or an envelope with a lipid bilayer. Polyethylene glycol can be chemically modified, so that it is bound covalently to specific —SH groups on the surface of the particle. The immunogenicity of the therapeutic agent, that is the directly inserted proteins or from the therapeutic nucleic acids transcribed and/or translated proteins, can be reduced with a fusion of 35 to 40 GA-(glycine-alanine)-repetitive sequences. GA-rich sequences naturally occur in the EBNA1 protein of the human Epstein-Barr virus and protect the viral protein from a degradation by the cellular proteasome and a presentation on class 1 MHC receptors. This safety function, in turn, can be performed for the different proteins and peptides used as a part of the mosaic-like vector system, with the in vitro assembling playing a positive role here.

### EXAMPLES

The following examples show applications of the invention, however, they shall not limit the area of protection of the invention. In the examples of the description the following figures are referred.

FIG. 1 is a schematic representation of the invention with possible forms of assembly. A capsid consisting of identical subunits modified at least twice or different partial units (components), is built up in a mosaic-like fashion. The assembled capsid can show certain properties, chosen before, which make it appear suitable, for example, for gene transfer.

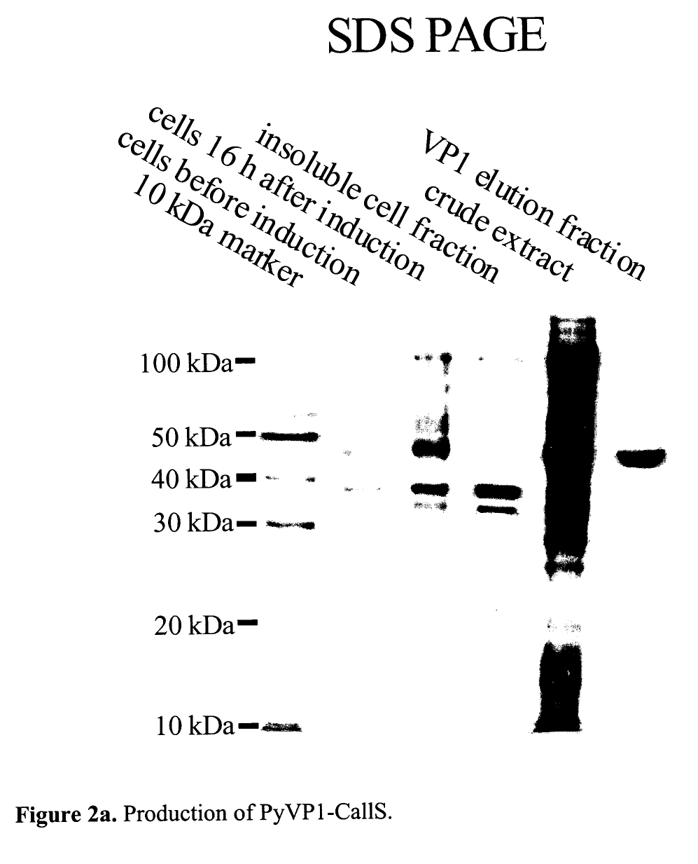

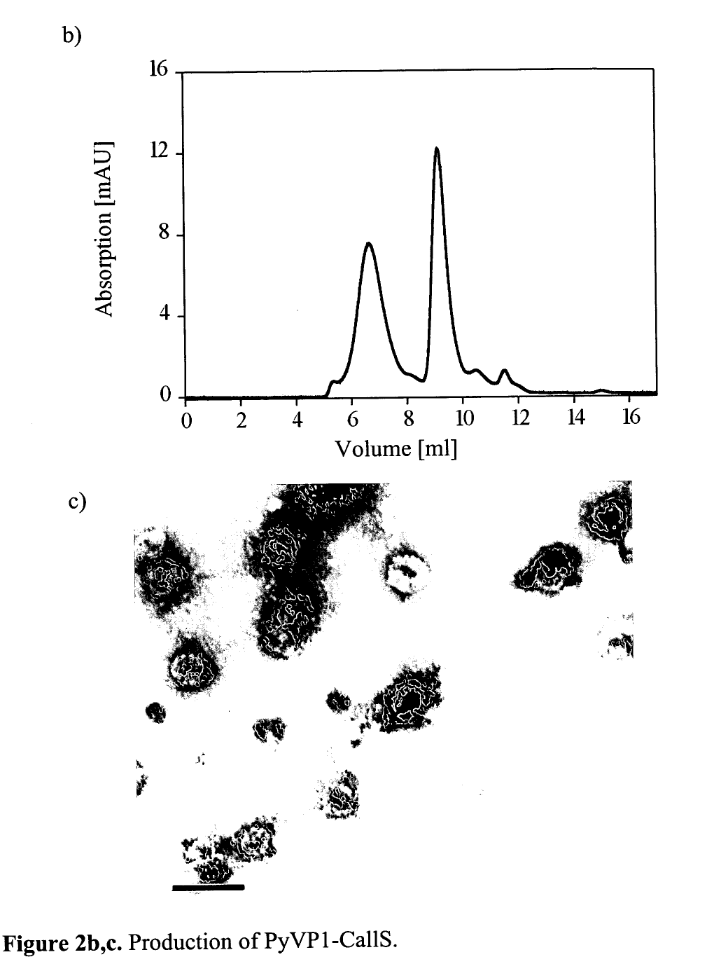

FIG. 2 shows the production of PyVP1-CallS. (a) Expression and purification of the variant PyVP1-CallS, according to the conditions indicated in example 1. (b) Gel filtration for detecting the assembly competence of the PyVP1-CallS variant. Capsids elute between 6 and 8 ml, free capsomeres between 9 and 10 ml. (c) Electron microscopic picture of capsids which consist exclusively of subunits of the variant PyVP1-CallS. Scaling bar: 100 nm.

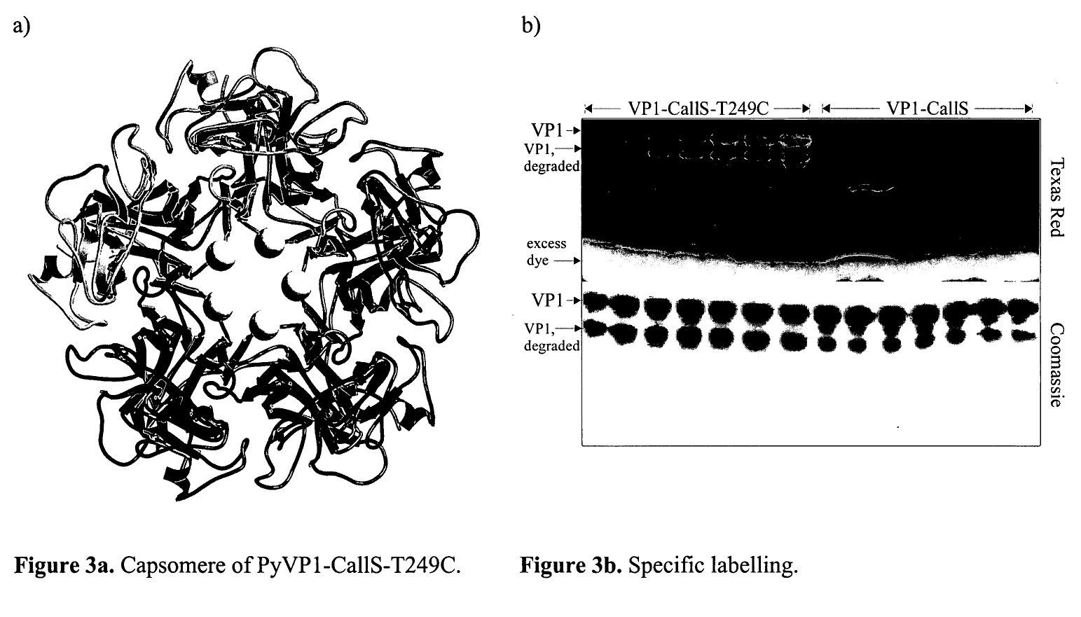

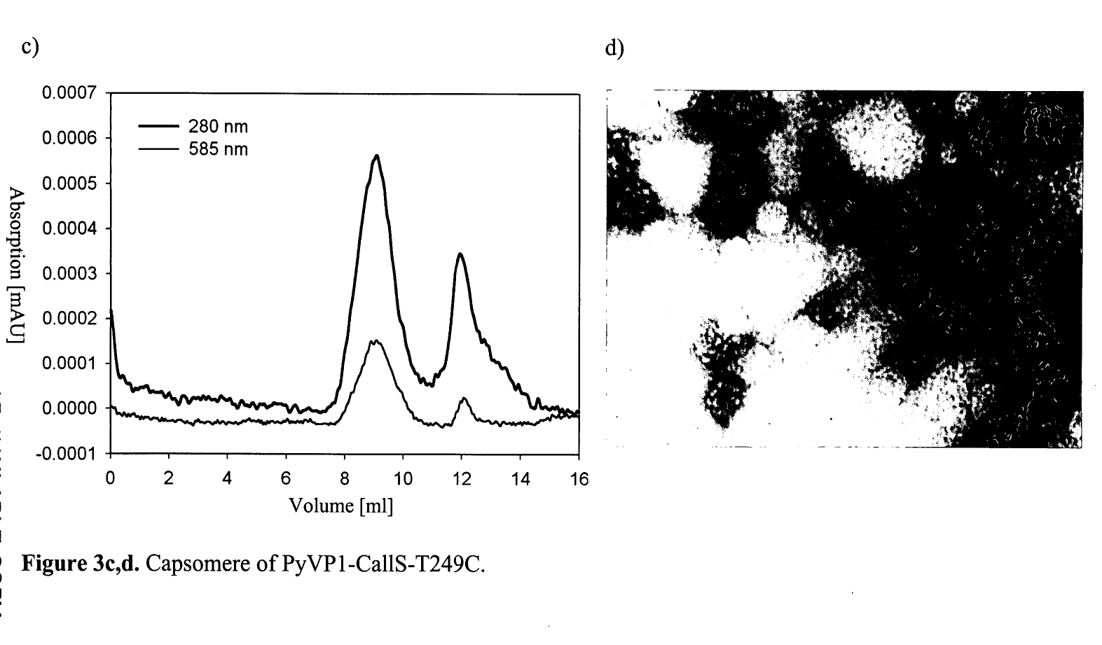

FIG. 3 shows capsomeres of PyVP1-CallS-T249C. (a) Top view on a three-dimensional structural representation of pentameric capsomeres of PyVP1-CallS-T249C (partial view). The amino acid position 249 in each subunit is marked by a ball; in this protected place, a specific, neutral labelling of the capsomeres is possible. (b) Specific labelling of the PyVP1-CallS-T249C protein (left half) at the unique cysteine opposite to the control PyVP1-CallS (right half). The staining caused by Coomassie dye (lower part) shows the presence of the proteins, but only the variant with cysteine at position 249 can be labelled by a dye like Texas Red (upper part). (c) Gel filtration for verification of the assembly competence of the PyVP1-CallS-T249C variant and integration of the dye into capsids. The capsids elute between 8 and 10 ml, free capsomeres between 11 and 12 ml. (d) Electron-microscopic picture of capsids, which consist exclusively of fluorescence-labelled subunits of the variant PyVP1-CallS-T249C. Scaling bar: 100 nm.

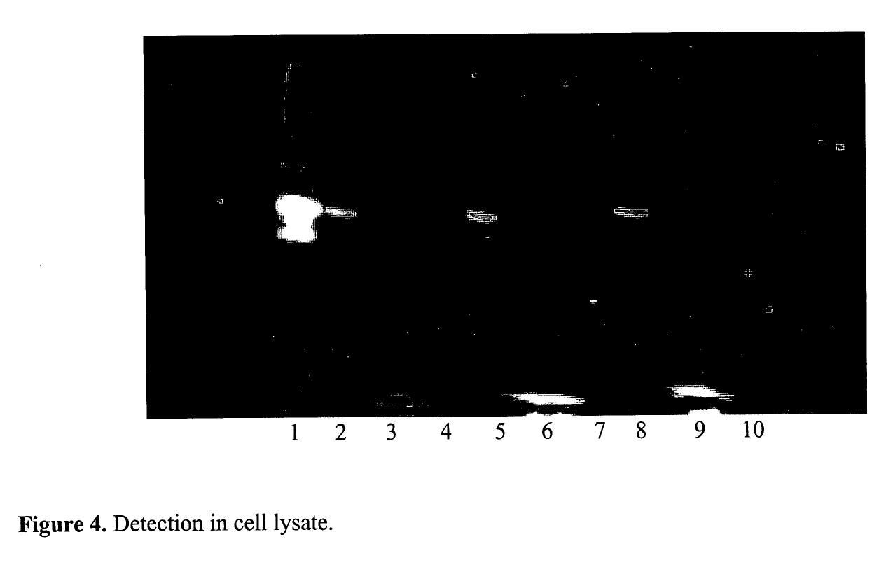

FIG. 4 shows the detection in the cell lysate. SDS gel (unstained) of cell lysate of eukaryotic C2C12 cells after incubation for 1 hour with fluorescent-labelled PyVP1-CallS-T249C capsids. The capsids taken up into the cells are degraded proteolytically to a large extent, the fluorescence dye is however clearly visible and therefore the uptake of the capsids into the cells is detectable. Lane 1, VP1-CallS-T249C labelled with Texas Red; lane 2, medium (supernatant) over the cells; lane 3: cell lysate with included particles; lane 4: wash fraction of the cells with PBS (no capsids included); lane 5 to 10: each lane analogous to lane 2 to 4 from parallel experiments of the same kind.

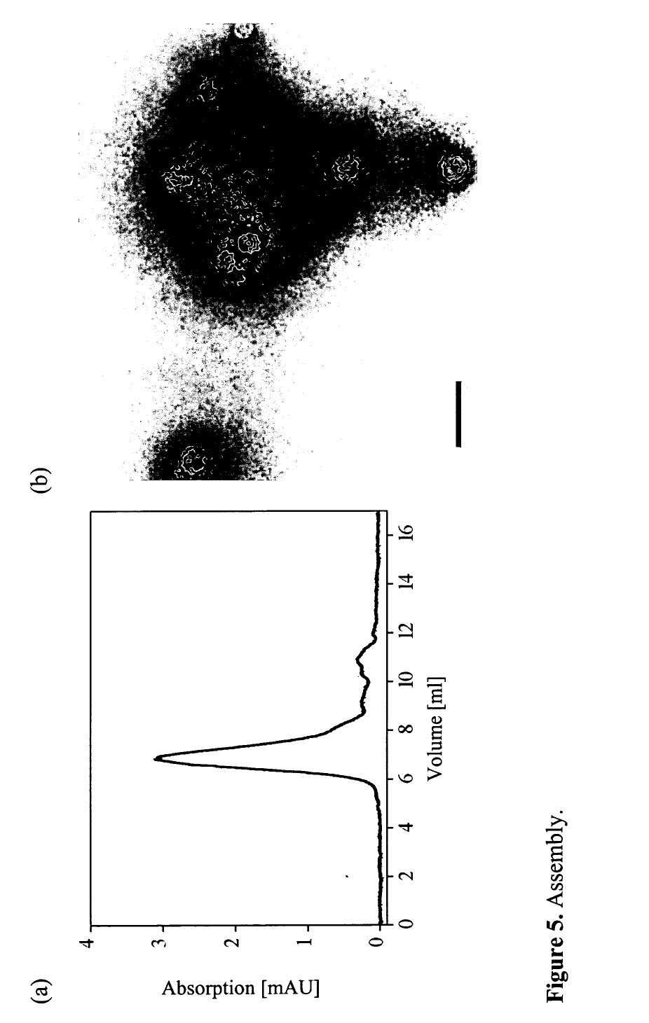

FIG. 5 shows the assembly. (a) Analysis of the assembly of the variant PyVP1-2C by means of gel filtration. The assembly of the capsomeres into capsids (elution at 6 to 8 ml) occurs completely, in contrast to the assembly of the PyVP1-CallS variant (FIG. 2b), free pentamers (9 to 10 ml) are not detectable anymore. (b) Electron-microscopic picture of the capsids, which are formed completely from PyVP1-2C .

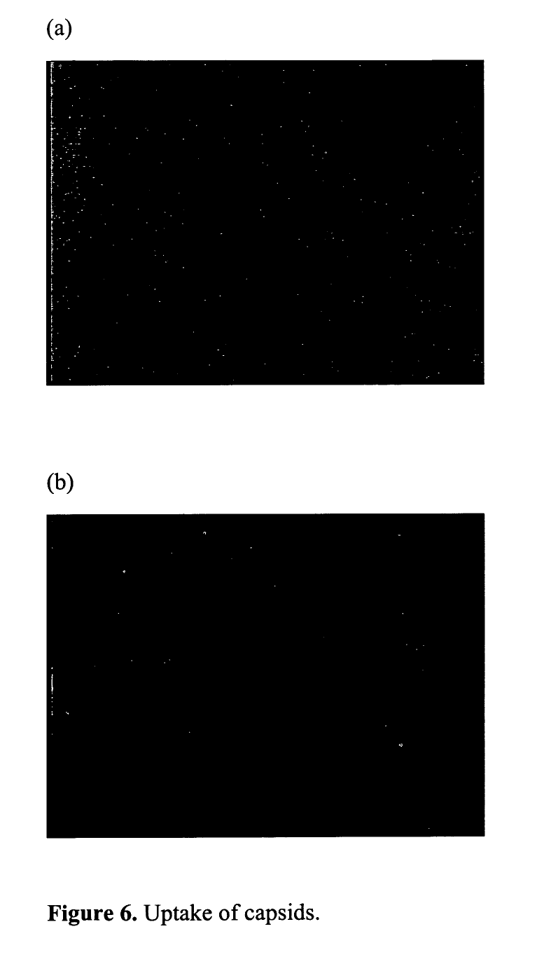

FIG. 6 shows the incorporation of capsids. (a) without RGD sequence motif, (b) with RGD sequence motif, in eukaryotic cells of the type Caco-2, otherwise under the same conditions. (a) Capsids of the type PyVP1-CallS-T249C are labelled with Texas Red and the uptake of the capsids into the cells are visualized in a fluorescence microscope. (b) Capsid uptake under identical conditions as in (a), however, the fluorescence-labelled capsids are of the type PyVP1-RGD148. These capsids are taken up significantly more efficiently into the cells due to the RGD motif.

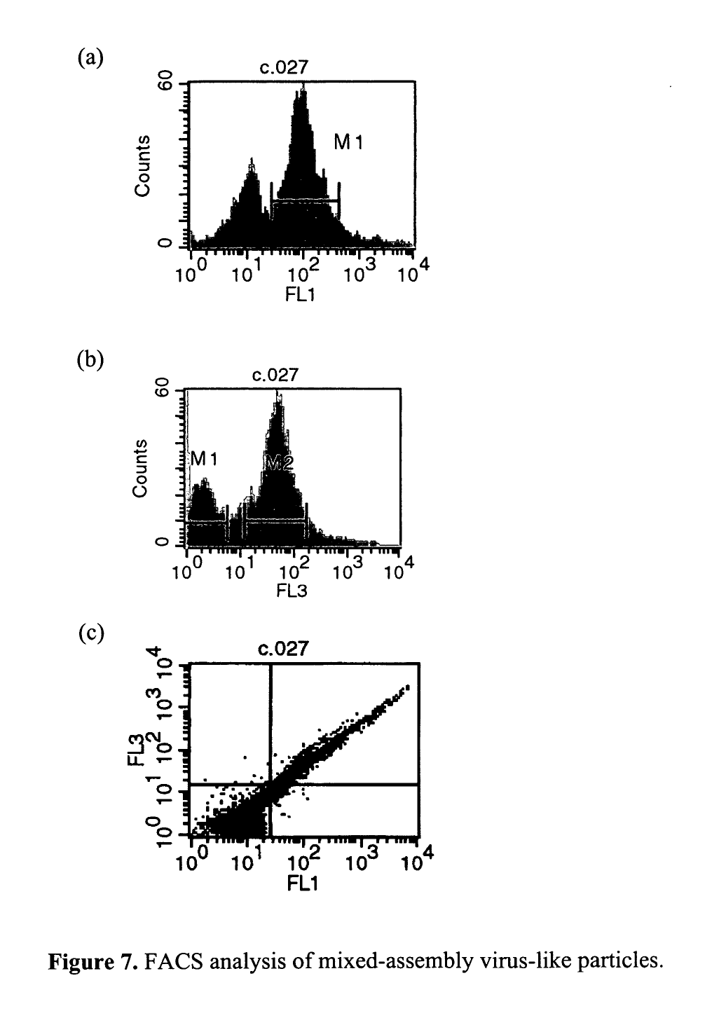

FIG. 7 shows a FACS analysis of differently labelled PyVP1 variants. Capsids from PyVP1-CallS-T249C are formed which consist of a species labelled with Fluorescein and another with Texas Red. The capsid population shows a clear Fluorescein fluorescence (M1 in a), as well as a Texas Red fluorescence (M2 in b). From the application of Fluorescein (FL1) compared to Texas Red (FL3) fluorescence, it becomes apparent that both dyes are localized on one particle (quadrant at the top on the right in c), particles that include only one dye are not created and therefore are not detected.

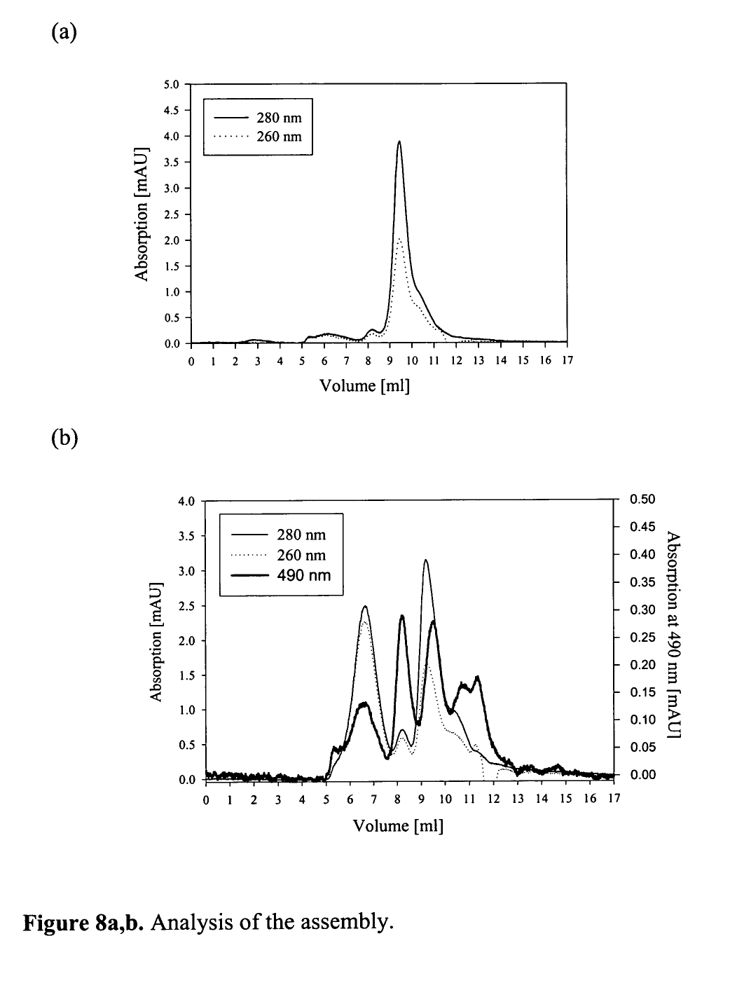

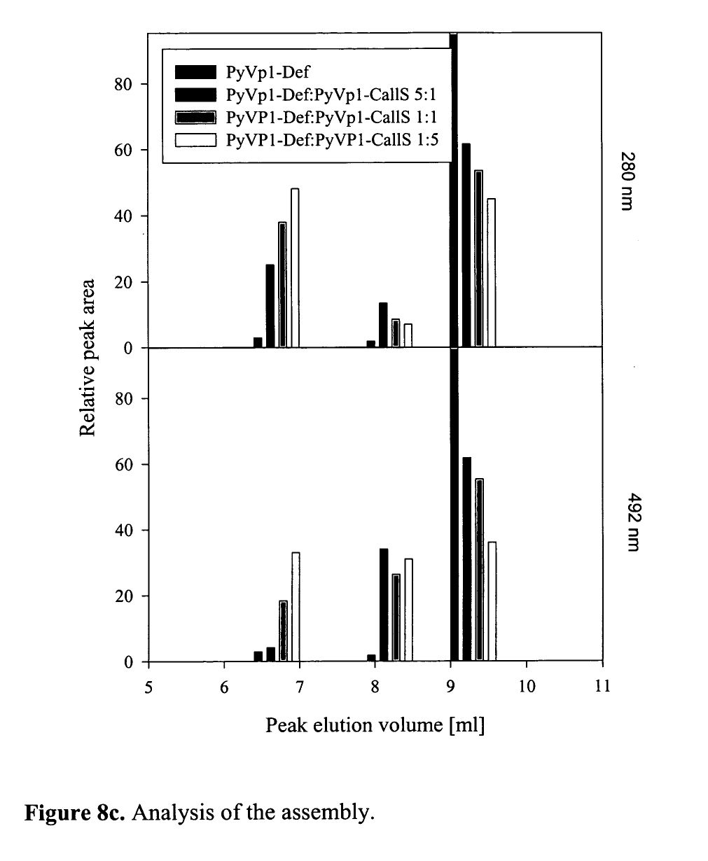

FIG. 8 shows an analysis of the assembly. (a) Gel filtration analysis of the assembly-deficient component PyVP1-Def. Under standard assembly conditions no capsids are formed, but only capsomeres are detected (elution at 9 to 10 ml). (b) Gel-filtration analysis of the mixed-assembled capsids, consisting of PyVP1-Def (fluorescent-labelled) and PyVP1-CallS (stoichiometric ratio 1:5). (c) Rates of inclusion of PyVp1-Def into capsids under different stoichiometric quantitative ratio of the capsomeres.

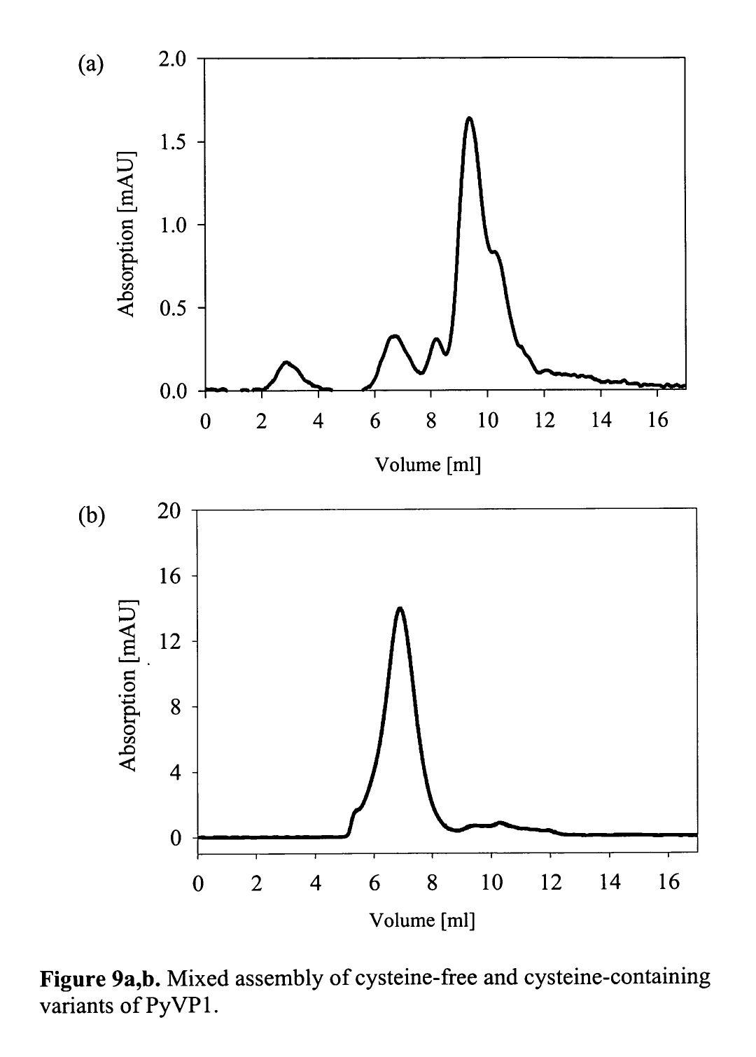

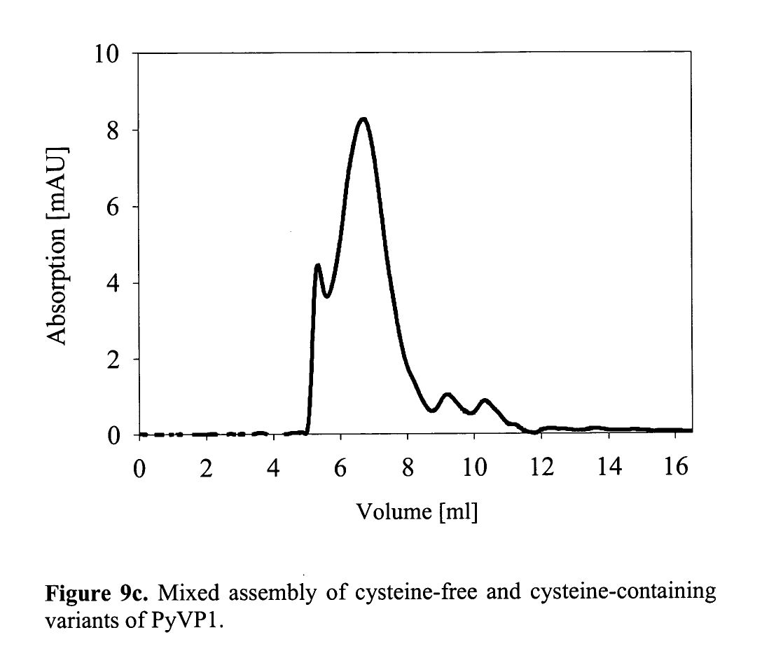

In FIG. 9, the mixed assembly of cysteine-free PyVP1-CallS-WW150 and cysteine-containing PyVP1-wt is shown. (a) The gel filtration analysis shows that the variant PyVP1-CallS-WW150 can only be assembled to about 15%. Capsids elute between 6 and 8 ml, free capsomeres between 11 and 12 ml. (b) The capsomeres of the variant PyVP1-wt form capsids quantitatively. (c) When assembling an equimolar mixture of both variants, quantitatively mixed capsids are formed, free capsomeres are not detected anymore. So, the property of a quantitative assembly of PyVP1-wt is transferred completely to the mixedly composed (mosaic-like) virus capsids.

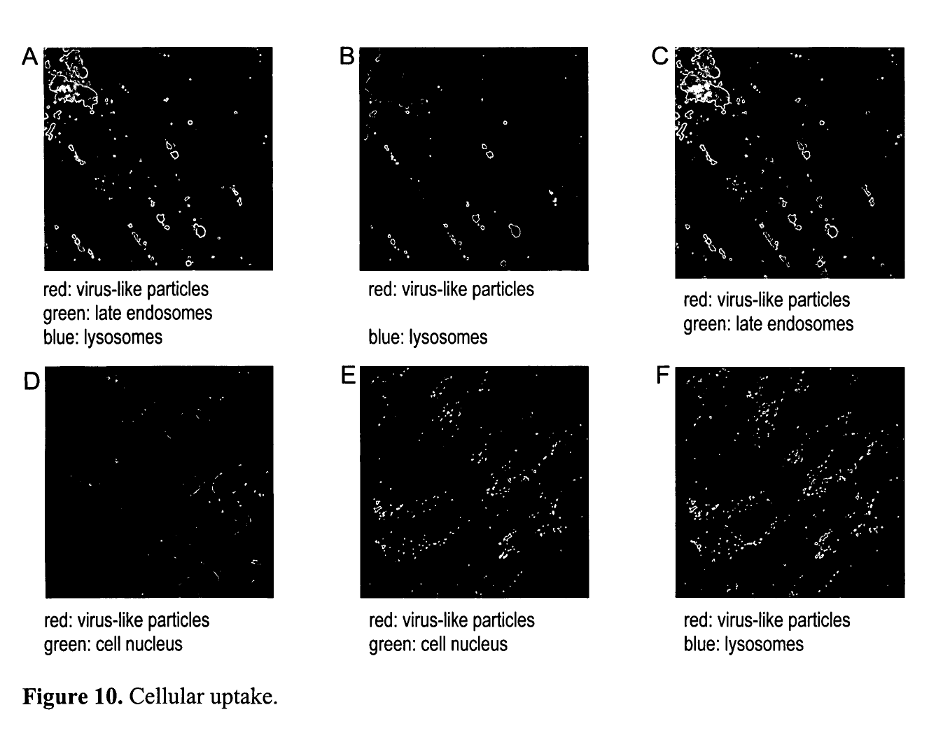

FIG. 10 shows the cellular uptake. Capsids from assembled PyVP1-CallS-T249C are incorparated into C2C12 cells and visualized by means of CLSM. In addition to the staining of the capsids (red, dye Texas Red, Molecular Probes), late endosomes (green, dye Fluorescein-Dextran, 70 kDa, Molecular Probes), nuclei (green, dye SYTO-16, Molecular Probes) and lysosomes (blue, dye LysoSensor Blue-Yellow, Molecular Probes) are shown. (a) to (c), localization of the capsids 15 min after uptake; (d) to (f), localization of the capsids 60 min after uptake. The capsids are included into the cells via endocytosis, pass through early and late (after 15 min) endosomes, and are finally enriched in lysosomes (60 min).

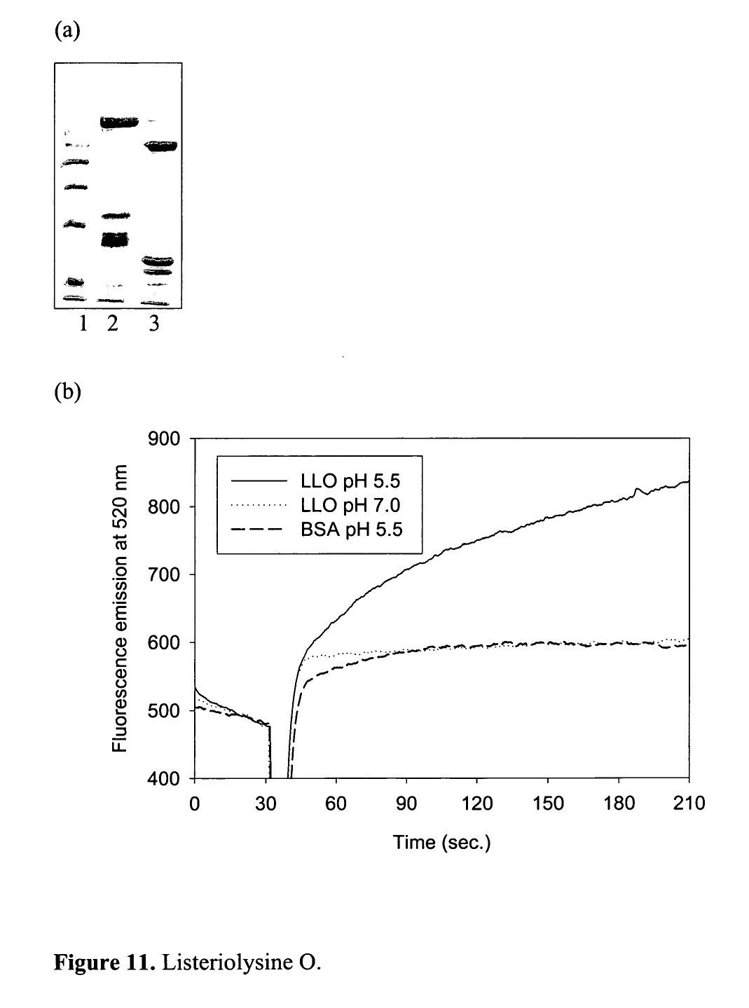

FIG. 11 shows the protein listeriolysine O. (a) Purification of the protein listeriolysine O (LLO) from Listeria monocytogenes. Lane 1, molecular mass standard (10 kDa ladder); lane 2, purified fusion protein of LLO and cellulose-binding domain according to example 8; lane 3, cleavage of the fusion protein with enterokinase and release of LLO. (b) Activity of the LLO protein, shown by the time course of the release of a fluorescence dye (Calcein, Sigma) from cholesterol-containing liposomes after adding LLO and lowering the pH value below pH 6.0. In control experiments, BSA as well as LLO were used at pH 7.0; these do not induce a release of the fluorescence dye.

### Example 1

Production, Assembly and Characterization of Cysteine-Free Coat Protein PyVP1 (PyVp1-CallS Variant)

The viral coat protein used in the given example is derived from the polyomavirus VP1 protein pentameric in solution, which can easily be assembled in vitro to an envelope according to the state of the technology. In this example, a polyomavirus variant is produced, which does not show any cysteines in the sequence; the six cysteines of the wild-type protein (Cys-12, Cys-16, Cys-20, Cys-115, Cys-274, and Cys-283) are replaced by serines by a site-directed mutagenesis process according to the state of the technology. This has the advantage among other things that the redox conditions of the solution do not have an influence on the state of the protein; this protein is therefore often easier to handle in a lot of applications.

The mutagenesis is carried out with the help of the QuickChange method (Stratagene), according to the manufacturer. For the mutagenesis, the following oligonucleodtides are used: C12S, C16S, C20S: 5′-GTC TCT AAA AGC GAG ACA AAA AGC ACA AAG GCT AGC CCA AGA CCC-3′, and 5′-GGG TCT TGG GCT AGC CTT TGT GCT TTT TGT CTC GCT TTT AGA GAC-3′, C115S: 5′-GAG GAC CTC ACG TCT GAC ACC CTA C-3′ and 5′-GTA GGG TGT CAG ACG TGA GGT CCT C-3′; C274S, C283S: 5′-GGG CCC CTC AGC AAA GGA GAA GGT CTA TAC CTC TCG AGC GTA GAT ATA ATG-3′ and 5 ′-CAT TAT ATC TAC GCT CGA GAG GTA TAG ACC TTC TCC TTT GCT GAG GGG CCC-3′.

The expression and purification of PyVP1-CallS occurs as fusion protein with a C-terminal fused intein domain and a chitin binding domain (CBD) attached to it. For this, a plasmid is produced first, which is based on the vector pCYB2 of the IMPACT system (New England Biolabs). Via the multiple cloning site of pCYB2, the DNA fragment is cloned using the restriction sites NdeI-XmaI (restriction enzymes by New England Biolabs) according to standard methods, this encodes for the PyVP1-CallS protein.

For the PCR of the DNA fragment, the following oligonucleotides are used: vp1NImp (5′-TAT ACA TAT GGC CCC CAA AAG AAA AAG C-3′), and vp1CImp (5′-ATA TCC CGG GAG GAA ATA CAG TCT TTG TTT TTC C-3′). With this PCR, the C-terminal amino acids of the wild-type VP1 protein are at the same time transformed from Gly383-Asn384 into Pro383-Gly384, as a C-terminal located asparagine is very unfavorable for the intein splitting system concerning the splitting properties. The mentioned exchanges do not affect the essential properties of the PyVP1protein for later assembly in the following.

The tac promoter of the pCYB2 vector delivers only little amounts of expression of the fusion protein, therefore, the fusion construct (PyVP1-CallS)-intein-CBD is isolated via another PCR from the pCYB2 vector and cloned into a a highly expressing pET vector with T7lac promotor (plasmid pET21a, Novagen) via NdeI-EcoRI restriction sites. The PCR occurs with the following oligonucleotides: vp1-NImp (5′-TAT ACA TAT GGC CCC CAA AAG AAA AAG C-3′), and 5 ′-ATA TGA ATT CCA GTC ATT GAA GCT GCC ACA AGG-3′.

The vector produced by this allows the expression of the fusion protein (PyVP1-CallS) Intein CBD with the help of the highly expressing T7lac promoters in E. coli BL21(DE3) cells (Novagen). For this, transformed cells are cultivated at 37° C. in 51—Erlenmeyer flasks, which contain 21 LB medium, until the OD₆₀₀ of the culture is 2.0 to 2.5. The induction of the protein expression occurs by 1 mM IPTG in the medium. Afterwards, the cultures are incubated at 15° C. for another 20 hours; the low temperature minimizes the elimination of the intein-part in the fusion protein under in vivo conditions. The cells are harvested by centrifugation, resuspended in 70 ml resuspension buffers (20 mM HEPES, 1 mM EDTA, 100 mM NaCl, 5% (w/v) glycerol, pH 8.0), and lysed by high-pressure homogenization. After centrifugating the crude extract for 60 min at 48 000 g, a clear cell extract is gained. This extract is put on a 10 ml chitin affinity matrix (New England Biolabs) with a flow rate of 0.5 ml/min at a temperature of 10° C. Afterwards, the column is washed with 3 column volumes of the resuspension buffer, 15 column volumes of a washing buffer of high ionic strength (20 mM HEPES, 1 mM EDTA, 2 M NaCl, 5% (w/v) glycerol, pH 8.0), and again 3 column volumes of the resuspension buffer; thereby, all unwanted E. coli host proteins are removed from the chitin matrix.

The elimination of the (PyVP1-CallS) capsomer, immobilized at the chitin matrix from the fusion protein with the help of the self-splicing intein activity, is induced in the resuspension buffer by a pulse (3 column volumes) with 50 mM dithiothreitol (DTT) each, 50 mM hydroxylamine, or 30 mM DTT together with 30 mM hydroxylamine. For this, the loaded chitin matrix is incubated for 14 hours at 10° C. with one of the mentioned solutions. The PyVP1-CallS protein is completely released and can be separated from the chitin matrix and the other components of the fusion protein adherent to the matrix by means of column chromatographical standard methods. Suitable for this, a linear salt gradient with a concentration between 0.1 and 2.0 M NaCl is used. According to the manufacturer, the regeneration of the chitin matrix occurs by washing the chitin material with 3 column volumes of a SDS-containing buffer (1% SDS (w/v) in resuspension buffer).

The assembly of the PyVP1-CallS proteins occurs first in analogy to conditions already described according to the state of the technology (cf. Salunke, Caspar & Garcea, Biophys. J. 56, S. 887-900, 1989). The virus-like capsids are maintained after dialysis of the protein against 10 mM HEPES, 50 mM NaCl, 0.5 mM CaCl₂, 5% glycerine, pH 7.2, for 72 hours at room temperature. With gel filtration (column TSKGel G5000PWXL and TSKGel G6000PWXL, TosoHaas), virus-like capsid coats can be detected and can be separated from free, non-assembled protein building blocks.

In the method described, the PyVP1-CallS protein is expressed as soluble pentamer and is assembly-competent. FIG. 2a shows a SDS gel for the representation of production und purification of PyVP1-CallS. FIG. 2b represents a gel filtration experiment that shows that the PyVP 1 -CallS protein can be assembled to capsid-like structures under suitable conditions. FIG. 2c describes the assembled capsids with the help of an electron-microsopic image.

The example shows that the PyVP1 wild-type protein can be modified, so that an assembly to capsid structures according to the state of the technology is also possible if there are no cysteines available in the protein coat. At the same time, the example shows the possibility of the efficient production of capsomeres with the help of an intein-based purification system.

### Example 2

Production, Assembly and Characterization of Fluorescence-Labellable Coat Protein PyVP1 (CallS-T249C Variant)

For the specific labelling of the capsomeres, a unique cysteine can be inserted into a special region of the protein. According to the tertiary structure of the protein represented in FIG. 3a, this is, for example, the position of the threonine 249, which is replaced by a cysteine. The mutagenesis occurs with the help of the QuickChange method (Stratagene) according to the manufacturer, using the oligonucleotides 5′-GGA CGG GTG GGG TGC ACG TGC GTG CAG TG-3′ and 5′-CAC TGG AGG CAC GTG CAC CCC ACC CGT CC-3′. Expression and purification of the protein are done in analogy to example 1.

The purified protein is labelled according to the manufacturer's protocol with the dyes Fluorescein-Maleimid or Texas Red-Maleimid (Molecular Probes). A specific coupling at the site of the cysteine 249 takes place; the specificity is shown in FIG. 3b. The protein can be assembled into capsids in analogy to example 1, as shown by gel filtration analyses (FIG. 3c) and electron microscope images (FIG. 3d).

The capsids labelled in this way are incubated on eukaryotic cells (C2C12 cells) for 1 hour. A 1000-fold excess of virus-like particles to cells is used. The adherent cells are washed with PBS after the incubation and are removed from the wall of the flask with the help of a cell scraper. Afterwards, the detached cells are mixed with SDS sample buffers and are heated up to 99° C. for 5 min. Then the cell lysate is separated via a SDS gel elelectrophoresis according to standard procedure. Here, the fluorescent-labelled protein components of the capsomeres become clearly visible without the usual staining of the gel. After the given time of incubation, an extensive degradation of the protein has already occurred in the cells (FIG. 4).

This example shows that a modified PyVP1-CallS protein can be produced with an additional unique cysteine in a safe position, can be labelled by fluorescent dyes and assembled into capsids. The capsids from this protein variant can be taken up into the interior of eukaryotic cells. The uptake can be detected by the fluorescent dye. The labelling does not influence the other properties of the protein.

### Example 3

Production, Assembly and Characterization of the Cysteine-Containing Coat Protein PyVP1 With and Without Fluorescence Labelling Options (2C/3C Variant)

The forming of an intrapentameric disulfide bridge between the amino acid positions 20 and 115 of PyVP1 can be advantageous for the assembly and the stability of the capsids. Therefore, a variant of PyVP1-CallS is produced which includes cysteines at both of the amino acid positions instead of the serines present. The mutagenesis is carried out according to the manufacturer with the help of the QuickChange method (Stratagene). For this, the following oligonucleotides are used: S20C: 5′-GCA CAA AGG CTT GTC CAA GAC CCG C-3′ and 5′-GCG GGT CTF GGA CAA GCC TTT GTG C-3′. The variant S115C is used as a template, which occurs as an intermediate product in the production of PyVP1-CallS according to example 1. The variant PyVP1-CallS-S20C-S115C produced in this way has two cysteines in suitable position for the intrapentameric disulfide bridge and is described as PyVP1-2C in the following.

Starting from this variant PyVP1-2C, another variant can be produced which includes an additional cysteine at position 249 and therefore is specifically and neutrally labellable in analogy to PyVP1-CallS-T249C from example 2. The mutaganesis occurs with the help of the QuickChange method (Stratagene) in analogy to example 2, and with the oligonucleotides described there.

For the production of both variants according to example 1, 30 mM DTT is used in the solvents as an additional additive in order to maintain the protein in the reduced state. The oxidation of the disulfide bridge in the capsomer occurs after the separation of the DTT in the scope of dialysis for assembling the capsomeres into capsids. FIG. 5 shows the assembly competence of the variant PyVP1-2C, in which the assembly incidentally occurs by means of dialysis in analogy to example 1.

A special feature of this variant compared to the PyVP1-CallS variant is the complete assembly of the capsomeres into capsids under oxidative conditions. Free, non-assembled capsomeres of the protein are not available anymore under the conditions mentioned. With the help of both of the variants described, a quantitative encapsidation of components into the virus-like particel can be achieved.

### Example 4

Production, Assembly and Characterization of the Coat Protein PyVP1 which Includes an Arginine-Glycine-Aspartate Sequence Motive at the Surface (PyVP1-RGD Variants)

Starting from PyVP1-CallS-T249C from example 2, two variants were produced which carry new sequences in a separate loop structure each on the outside of the capsid shell. A special feature of these new sequence segments is the appearance of a sequence Arg-Gly-Asp (RGD). With these variations, a cellular uptake mechanism for the artificial capsids shall be implanted which is comparable to the mechanism of adenoviruses. According to the state of the technology, it is known for this virus class that binding to integrin receptors on the cellular surface enables the uptake of the adenoviruses into the cells.

The insertion of the new sequence motifs is carried out between the sequence positions 148 and 149, on one hand, and between the amino acid positions 293 and 295, on the other hand. The corresponding areas are on the outside of the capsids according to the structure.

The insertion of a new loop segment with alternating flexible serine-glycine motifs at position 148/149 (for the following production of the variant PyVP1-RGD148) occurs with the help of the QuickChange method (Stratagene) by using the following oligonucleotides: 5′-CAA CAA ACC CAC AGA TAC AGT AAA CGG CAG CGG CAG CGG CAG CGG CAG CGG CAG TGC AAA AGG AAT TTC CAC TCC AGT G-3′ and 5′-CAC TGG AGT GGA AAT TCC TTT TGC ACT GCC GCT GCC GCT GCT GCC GCT GCC GCT GCC GTT TAC TGT ATC TGT GGG TTT GTT G-3′. For the insertion of an analogous loop segment at position 293/295 (for the following production of the variant PyVP1-RGD293), the following oligonucleotides are used: 5′-GAT ATA ATG GGC TGG AGA GTT ACC GGC AGC GGC AGC GGC AGC AGC GGC AGC GGC AGT GGC TAT GAT GTC CAT CAC TGG AG-3′ and 5′-CTC CAG TGA TGG ACA TCA TAG CCA CTG CCG CTG CCG CTG CTG CCG CTG CCG CTG CCG GTA ACT CTC CAG CCC ATT ATA TC-3′. In a second step, the oligonucleotides 5′-CGG CAG CGG CAG CGG CAG CGG TCG TGG CGA TAG CGG CAG CGG CAG CGG CAG TG-3′ and 5′-CAC TGC CGC TGC CGC TGC CGC TAT CGC CAC GAC CGC TGC CGC TGC CGC TGC CG-3′ are used in order to insert the Arg-Gly-Asp sequence into the newly created loop segments in both variants described. After this final cloning, both variants PyVP1-RGD148 and PyVP1-RGD293 are produced on a genetic level.

The production and purification of both protein variants occurred in analogy to example 1. The assembly of both variants into capsids is successful with the assembly conditions given in example 1, the capsomeres are native and assembly-competent. Furthermore, it is possible to label these variants with fluorescence dyes at the unique cysteine C249, in analogy to example 2. The assembled capsids consisting of fluorescent-labelled capsomeres can be incubated on eukaryotic cells (type Caco-2). The uptake of the capsids into the cells can be followed via the fluorescence dye with the help of a fluorescence microscope; a fixation of the cells is not necessary for that. As FIG. 6 shows, an uptake of the capsids into the cells occurs. The PyVP1-RGD148 variant (FIG. 6b) is here taken up more efficiently than the comparable control variant PyVP1-CallS-T249C (FIG. 6a) without the RGD sequence motif. Therefore, the implanted RGD motif induces a capsid uptake via an efficient way by integrin-receptor mediated endocytosis. Moreover, the example shows that a control of the uptake of the capsids into cells is possible using suitable components.

### Example 5

Production, Assembly and Characterization of Coat Protein PyVP1 that Shows a Change in the Natural Sialyllactose Binding Site (PyVP1-R78W Mutant)

Another variant is produced on the basis of the variant PyVP1-3C which contains a mutation of the amino acid arginine 78 to tryptophan (PyVP1-3C-R78W). The position of the arginine 78 is considerably involved in the binding of the natural virus to sialyllactose on the surface of the cell, which is the natural receptor of the polyomavirus. The suppression of this interaction by the mutation R78W, i.e. an exchange of the arginine for a structurally incompatible tryptophan, should prevent an uptake of the virus particles into the target cells via the natural receptor binding.

The given mutation is carried out according to the manufacturer with the QuickChange method (Stratagene). Therefore the following oligonucleotides are used: 5′-CTA TGG TTG GAG CTG GGG GAT TAA TTT G-3′ and 5′-CAA ATT AAT CCC CCA GCT CCA ACC ATA G-3′. The production and purification as well as the assembly of the resulting PyVp1-R78W variant occurs in analogy to example 1.

Similar to the other variants documented, the variant PyVP1-R78W is able to assemble into capsids. The example shows that the cell tropism of the capsids can be manipulated by variation of the surfaces of the capsid structures. By this, new cell tropisms can be inserted as well as present cell tropisms can be eliminated.

### Example 6

Production and Characterization of Mixed Capsids I

The production of mixed capsids, i.e. particles, which are built up in a mosaic-like fashion from several different molecular substances, is a particularly important feature of this invention. For the verification of mixed capsids, built up from different coat proteins, the variant PyVP1-CallS-T249C of the coat protein is coupled to the unique cysteine 249 with the fluorescent dye Fluorescein-Maleimid in one approach, and with Texas Red-Maleimid in a second approach, as described in example 2. The differently labelled capsids are mixed in equimolar ratio and are subsequently assembled. The analysis of the capsid formation occurs by means of flow cytometry (FACS). This technique enables the detection of different fluorescence types within a single particle. FIG. 7 shows the analysis of equimolarly assembled capsids. A population of fluorescence-labelled capsids and free non-assembled capsomeres (FIG. 7a/b) appears. When showing Fluorescein fluorescence versus Texas Red fluorescence in a graph (FIG. 7c), a population of particles is observed which carries both fluorescences at the same time. Particles that are labelled with only one dye cannot be detected as they are obviously not formed.

This example shows that polyomavirus VP1 coat proteins which show different properties can be assembled in a mosaic-like fashion, so that virus capsids are formed, which specifically show the properties of both coat proteins. Apart from this, a method for a highly sensitive determination and analysis of the composition of the virus capsids is shown.

### Example 7

Production and Characterization of Mixed Capsids II

For further demonstration of the advantages of the invention, a variant of PyVP1 is produced which is completely assembly-deficient. For this, an artificial peptide sequence is inserted (sequence GSGSG WTEHK SPDGR TYYYN TETKIQ STWEK PDDGS GSG) between the positions 293 and 295 of the amino acid sequence of PyVP1-CallS-T249C according to the state of the technology with the help of PCR. The production and purification of the variant occurs according to the explanations in example 1. The produced variant PyVP1-Def is a native, pentameric protein. However, it is completely assembly-deficient and cannot form virus-like capsids under the standard conditions for assembly from example 1. This assembly deficiency is shown with gel filtration analysis (FIG. 8a).

The assembly-deficient variant PyVP1-Def is labelled with the fluorescent dye Fluorescein-Maleimid (Molecular Probes) according to the example 2 via the unique cysteine at position 249. Afterwards, an assembly in the presence of the variant PyVP1-CallS occurs in different stoichiometric ratios of both components. The assembly is carried out by means of dialysis according to the example 1. The measurement of the absorption of the fluorescein at 490 nm in a gel filtration analysis (FIG. 8c) shows that PyVP1-Def is built into the assembling capsids. The variation of the stoichiometric ratios of both capsid components during the assembly (FIG. 8c) demonstrates that an inclusion of the assembly-deficient variant PyVP1-Def into the mixed capsids occurs in proportion to the mass ratios of the variants.

This example also shows that mixed capsids from different variants of polyomavirus VP1 can be formed under assembly conditions. Furthermore, the capsomeres built into the capsids reflect the stoichiometric mass ratios of the starting conditions. The described method can also be used to integrate capsomeres into the capsid structures which are otherwise assembly-deficient.

### Example 8

Production and Characterization of Mixed Capsids III

The assembly of cystein-free VP1 variants, like for example PyVP1-CallS from example 2, can have the disadvantage that not all of them, for example 50% of the capsomeres used, form capsids. Apart from that, these capsids can be relatively instable und dissemble partly after isolation. This disadvantage can be compensated by a mixed assembly with cysteine-containing variants.

The cysteine-free variant PyVP1-CallS-WW150 shows a reduced assembly ability of about 15% compared to 50% using PyVP1-CallS (FIG. 9a), whereas cysteine-containing capsomeres of the variant VP1-wt completely assemble into virus capsids under suitable conditions similar to PyVP1-2C and PyVP1-3C from example 3 (FIG. 9b). An equimolar mixture of the variants PyVP1-CallS-WW150 and PyVP1-wt under assembling conditions leads to mosaic-like mixed virus-like capsids. This becomes apparent by the fact that the mixture assembles completely and no free capsomeres can be detected anymore (FIG. 9c).

This example also verifies the formation of virus capsids built in a mosaic-like fashion. Furthermore, the possibility to combine cysteine-containing with cysteine-free variants is demonstrated, in which the properties of the cysteine-containing capsomeres, a complete assembly into stable virus capsids, is transferred to the whole capsid. This effect enables the modification of otherwise cysteine-free capsomeres highly specifically at a cysteine residue inserted at a defined site (for example using PyVP1-CallS) and to insert this new function into a virus capsid, in which the disadvantages of cysteine-free assembly (less assembly efficiency, more instable capsids) are avoided.

### Example 9

Transfection of Cells with Virus-Like Capsids

The variant PyVP1-CallS-T249C can be produced, assembled and fluorescence-labelled according to example 2. The labelled capsids can be shown intracellulary with the help of confocal laser scan microscopy (CLSM) after uptake into eukaryotic cells of the type C2C12. Therefore, this PyVP1 variant offers the possibility to analyze efficiency and uptake mechanism of homogeneously or heterogeneously (mixed) assembled capsids, built up in a mosaic-like fashion. Therefore, fluorescence-labelled PyVP1-CallS-T249C is built into the capsid particles.

In FIG. 10, a series of experiments for the uptake of capsids, consisting of assembled PyVP1-CallS-T249C, into C2C12 cells is demonstrated. In addition to the staining of the capsids (red, dye Texas Red, Molecular Probes), late endosomes (green, dye Fluorescein-Dextran 70 kDa, Molecular Probes), cellular nuclei (green, dye SYTO-16, Molecular Probes), and lysosomes (blue, dye LysoSensor Blue-Yellow, Molecular Probes) are visualized. The capsids are taken up into the cells via endocytosis, go through early and late (after 15 min) endosomes, and are finally enriched in lysosomes (60 min).

The example demonstrates that an analysis of the properties of the components is possible with the help of the variants described before and these analyses may also comprise cellular localizations und active mechanisms of the capsids. Therefore, a possibility is shown to analyze and describe the biological properties of the artificially produced, mosaic-like capsids by using labellings, with the labelling itself being neutral and not having an influence on these properties.

### Example 10

Transfection of Cells With Complexes from DNA and Virus-Like Capsids

For demonstrating the transport of DNA by virus-like capsids into eukaryotic cells, 100 μl of a solution of the protein PyVP1-3C (1 mg/ml in 20 mM HEPES pH 7.2, 100 mM NaCl, 10 mM DTT, 1 mM EDTA, 5% glycerol) are mixed with 7.5 μl of a solution of the plasmid pEGFP-N1 (Clontech) as well as 100 μl dialysis buffer (20 mM sodium acetate, pH 5.0, 100 mM NaCl, 1 mM CaCl₂, 5% glycerol) and are dialysed for 4 days at room temperature with frequent buffer changes against dialysis buffer. For producing the transfection medium, 100 μl of this reaction mixture is mixed with 300 μl Dulbecco's Modified Eagle Medium (DMEM)+1 μM chloroquine. For the transfection test, NIH-3T3 cells are used, which were seeded in a 12 well plate in a density of 10 000 cells per well the day before. For the transfection, the cells are washed with PBS (phosphate buffered saline) and are mixed with 400 μl transfection medium per well. The cells are incubated for 1 h at 37° C. and with 5% CO₂, afterwards washed with PBS and incubated with complete medium (DMEM+10% FBS) for another 48 h at 37° C. and 5% CO₂. After this period of time, a successful transfection can be detected by the expression of a reporter gene. The plasmid pEGFP-N1 used here allows the expression of GFP (green fluorescent protein), which can be detected due to its green fluorescence in the cells. With the protocol described here, about 20 cells per well on average can be identified which produce the reporter gene GFP unambiguously.

This example shows that DNA can be successfully transported into eucaryotic cells with the transport system described here, consisting of PyVP1-3C. In addition, the inserted DNA can be released in the cells and a reporter gene can be expressed correctly.

### Example 11

Production and Characterization of Listeriolysine O (LLO)

As it becomes apparent in example 7, a large amount of the produced virus-like capsids is enriched in lysosomes after their uptake into eukaryotic cells and are finally lysed. A release of the particles from the endosomal compartiment can be induced by adding cytolysines. A model for this is the listeriolysine O (LLO) from the organism Listeria monocytogenes.

The LLO gene is amplified according to the standard method from a fragment of genomic DNA of Listeria monocytogenes with the help of PCR. For this, a cloning in the vector pTIP is carried out with the help of the oligonucleotides 5′-TAT AGA CGT CCG ATG CAT CTG CAT TCA ATA AAG AAA ATT-3′ and 5′-TAC TTA AGG CTG CGA TTG GAT TAT CTA CAC TAT TAC TA-3′. This vector pTIP is a derivative of the intein expression vector, documented in example 1, on the basis of pET21a, with additionally inserted proline-rich sequences. The vector is constructed, so that a proline-rich sequence can be fused to the 5′- or 3′ end of the gene, alternatively, inserted via a multiple cloning site. The proline-rich sequence mainly includes the sequence Pro-Pro-Pro-Pro-Pro-Pro-Pro-Pro-Leu-Pro.

In a second PCR, the gene fragment is amplified from the pTIP vector and cloned into a pET34b vector (Novagen). For this, the oligonucleotides 5′-GCC GCC ACC TCC ACC GCC AC-3′ and 5′-ATT AGG GTT CGA TTG GAT TAT CTA CAC TAT TAC-3′ are used. The vector is cut with Srf I (Stratagene) blunt end and the DNA fragment is ligated blunt end into the vector pET34b. The produced construct allows an expression of the LLO protein labelled by means of proline-rich sequence as N-terminal fusion protein with a cellulose binding domain. This binding domain can be proteolytically separated after successful affinity purification with the help of enterokinase. The production of the fusion protein occurs by cultivation of transformed BL21(DE3) cells at 25° C. after induction with 1 mM IPTG. The cell homogenisation occurs according to example 1. As resuspension buffer, 20 mM HEPES, 200 mM NaCl, pH 7.0, is used here. For removing bacterial DNA, the cell extract is mixed with 5 mM MgCl₂ and 0.1 U Benzonase and incubated for 30 min at 25° C.

Afterwards, the purification of the fusion protein occurs by putting the cell extract on a cellulose matrix (Novagen) according to the manufacturer. The elution of the fusion protein occurs with 1 column volume of ethylene glycol (Merck). The eluted protein is dialyzed immediately against resuspension buffer. The elimination of the cellulose binding domain from the fusion protein is carried out according to the manufacturer using enterokinase.

FIG. 11a shows a SDS electrophoresis gel which documents the production and purification of the LLO. The activity of the protein is demonstrated in FIG. 11b. The protein can induce pores into cholesterol-containing lipid bilayer membranes under suitable solvent conditions (pH<6.0). This is shown on synthetic, cholesterol-containing liposomes, which are produced according to a standard method. Here, a fluorescence dye (Calcein) is released from the synthetic liposomes and a measurable increase of the fluorescence signal in the solution occurs (FIG. 11b).

The example shows that the protein LLO can be produced recombinantly in active form. Moreover, it is shown that LLO can dissolve biological membranes as they occur in endodomes. In connection with the capsids from example 1 to 7, this property can be used for the release of capsids taken up into endosomes.

1-21. (canceled)

22. Transport system for molecular substances, containing recombinantly produced partial units based on amino acids, including:
at least two partial units modified once differently from each other, and/or
one or several partial units modified differently at least twice, and
alternatively unmodified partial units,
with the partial units being able to assemble to a transport system like a mosaic and molecular substances can be encapsidated into the transport system, and
where the modified partial units are partial units from the Polyomavirus VP1 protein which contain as a modification one or more amino acids, peptide or protein sequences or protein domains in a loop region at the outside of the protein at amino acid position 148, if the VP1 protein units are cysteine-free or contain cysteines, or the modified VP1 protein units contain such a modification at amino acid position 148 or 293, if the VP1 protein partial units contain the cysteines from the wild-type VP1 protein.

23. Transport system according to claim 22, wherein single-stranded or double-stranded DNA, single-stranded or double-stranded RNA, peptides, peptide hormones, proteins, protein domains, glycoproteins, ribozymes, PNA (peptide nucleic acid), pharmaceutical agents, nucleotides, hormones, lipids or carbohydrates can be encapsidated as molecular substances.

24. Transport system according to claim 22, wherein single-stranded or double-stranded DNA, single-stranded or double-stranded RNA, encapsidated in the transport system, code for proteins that are provided with a signal sequence so that the proteins are transported either into the nucleus, the mitochondria, the endoplasmatic reticulum, or out of the cell.

25. Transport system according to claim 22, wherein the substances encapsidated in the transport system are supplied with a signal molecule, so that they are transported either into the nucleus, the mitochondria, the endoplasmatic reticulum, or out of the cell.

26. Transport system according to claim 22, wherein the proteins encoded by the encapsidated single-stranded or double-stranded DNA, or single-stranded or double-stranded RNA, or encapsidated proteins or proteins that are a component of the coat show a reduced cellular degradation rate by the fusion of sequences that are rich in glycine and alanine at the aminoterminal end, by which a stimulation of the immune system in living organisms does not occur or is reduced.

27. Transport system according to claim 22, wherein the transport system is covered by a coat that protects it against an immune response of the organism.

28. Transport system according to claim 27, wherein the coat consists of polyethylene glycol or is carried out in the form of a synthetically produced lipid membrane.

29. Transport system according to claim 22, wherein the recombinantly produced partial units are modified by point mutations or by insertion, removal or change of one or several peptide or protein sequences or protein domains at the terminus/the termini and/or in the sequence of the partial unit, or that the recombinantly produced partial units are modified in such a way that they are taken up efficiently and/or directed against target cells, and/or the molecular substances can be better bound and associated to the partial units.

30. Transport system according to claim 22, wherein the transport system shows at least one partial unit which is modified in an area that is located at the inside of the transport system, so that the molecular substance(s) can be better bound or associated to the partial units.

31. Transport system according to claim 22, wherein the recombinantly produced partial units are modified by point mutations and/or by coupling of peptides, peptide hormones, proteins, protein domains, glycoproteins, lipids or carbohydrates in such a way that they can be taken up specifically in selected cell types.

32. Transport system according to claim 22, wherein the transport system shows at least one partial unit that carries an RGD sequence in a loop structure, which is located at the outside of the transport system, which enables an uptake of the transport system into the target cell by means of integrin receptor-mediated endocytosis.

33. Transport system according to claim 22, wherein the recombinantly produced partial units are modified at least with one or several proteins, one or several protein domains, one or several peptides, one or several dendrimers, or hydrophobic or basic polymers in such a way that the transport system or parts of it can pass through the endosomal membrane.

34. Transport system according to claim 33, wherein bacterial cytolysins or viral proteins are used as proteins and translocation domains of bacterial toxins are used as protein domains.

35. Transport system according to claim 22, wherein the recombinantly produced partial units can be labelled with a fluorescence dye, oligonucleotides, peptides, peptide hormones, lipids, fatty acids or carbohydrates.

36. Method for the production of transport systems according to claim 22 with the following steps:
recombinant expression of the partial units from the polyomavirus VP1 protein,
release of the partial units by lysis of the host cells,
creating contact between the partial units in the desired stoichiometric relations in order to compose/to assemble the transport system in a mosaic-like fashion, and
optionally creating contact with molecular substances, either before or during the assembly, in order to encapsidate the molecular substances into the transport system.

37. Method according to claim 36, wherein the recombinantly produced modified partial units are assembled using appropriate solvent conditions in selected stoichiometric relations, by which the functional features of the molecular transport system can be checked/determined/controlled.

38. Use of the transport systems according to claim 22 for the transfer of molecular substances in cells.

39. Use of the transport systems according to claim 22 for the transfer of DNA in eukaryotic cells.

40. Use according to claim 38, where cytolysines, preferentially listeriolysin O, are added in order to release the transport systems from the cellular lysosomes.

41. Use according to claim 38, wherein partial units of the molecular transport system are la-belled with fluorescence dyes, so that the molecular transport systems can be localized inside the cell.

42. Pharmaceutical composition, containing one or several transport systems according to claim 22, together with usual pharmaceutically acceptable additives and adjuvants.


SEQUENCE LISTING (49 sequences)

SEQ ID NO: 1 - DNA, 1152 bp, Artificial Sequence
  Description of Artificial SequencePyVP1-CallS variant of polyoma virus coat protein VP1
atggcccccaaaagaaaaagcggcgtctctaaaagcgagacaaaaagcacaaaggctagcccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacgtctgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgacaaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 2 - protein, 384 aa, Artificial Sequence
  Description of Artificial SequencePyVP1-CallS variant of polyoma virus coat protein VP1
MAPKRKSGVSKSETKSTKASPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTSDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTTTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 3 - DNA, 1152 bp, Artificial Sequence
  Description of Artificial SequencePyVP1-CallS-T249C variant of polyoma virus coat protein VP1
atggcccccaaaagaaaaagcggcgtctctaaaagcgagacaaaaagcacaaaggctagcccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacgtctgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 4 - protein, 384 aa, Artificial Sequence
  Description of Artificial SequencePyVP1-CallS-T249C variant of polyoma virus coat protein VP1
MAPKRKSGVSKSETKSTKASPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTSDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 5 - DNA, 1152 bp, Artificial Sequence
  Description of Artificial SequencePyVP1-2C variant of polyoma virus coat protein VP1
atggcccccaaaagaaaaagcggcgtctctaaaagcgagacaaaaagcacaaaggcctgtccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacctgtgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgacaaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 6 - protein, 384 aa, Artificial Sequence
  Description of Artificial SequencePyVP1-2C variant of polyoma virus coat protein VP1
MAPKRKSGVSKSETKSTKACPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTCDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTTTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 7 - DNA, 1152 bp, Artificial Sequence
  Description of Artificial SequencePyVP1-3C variant of polyoma virus coat protein VP1
atggcccccaaaagaaaaagcggcgtctctaaaagcgagacaaaaagcacaaaggcctgtccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacctgtgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 8 - protein, 384 aa, Artificial Sequence
  Description of Artificial SequencePyVP1-3C variant of polyoma virus coat protein VP1
MAPKRKSGVSKSETKSTKACPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTCDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 9 - DNA, 1188 bp, Artificial Sequence
  Description of Artificial SequencePyVP1-RGD148 variant of polyoma virus coat protein VP1
atggcccccaaaagaaaaagcggcgtctctaaaagcgagacaaaaagcacaaaggctagcccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacgtctgacaccctacaaatgtgggaggcagtctcagtgaaaaccgaggtggtgggctctggctcactgttagatgtgcatgggttcaacaaacccacagatacagtaaacggcagcggcagcggcagcagaggcgacagcggcagtgcaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 10 - protein, 396 aa, Artificial Sequence
  Description of Artificial SequencePyVP1-RGD148 variant of polyoma virus coat protein VP1
MAPKRKSGVSKSETKSTKASPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTSDTLQMWEAVSVKTEVVGSGSLLDVHGFNKPTDTVNGSGSGSRGDSGSAKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 11 - DNA, 1200 bp, Artificial Sequence
  Description of Artificial SequencePyVP1-RGD293 variant of polyoma virus coat protein VP1
atggcccccaaaagaaaaagcggcgtctctaaaagcgagacaaaaagcacaaaggctagcccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacgtctgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggagagttaccggcagcggcagcggcagcggtcgtggcgatagcggcagcggcagcggcagtggctatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 12 - protein, 400 aa, Artificial Sequence
  Description of Artificial SequencePyVP1-RGD293 variant of polyoma virus coat protein VP1
MAPKRKSGVSKSETKSTKASPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTSDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWRVTGSGSGSGRGDSGSGSGSGYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 13 - DNA, 1152 bp, Artificial Sequence
  Description of Artificial SequencePyVP1-R78W (PyVP1-3C-R78W) variant of polyoma virus coat protein VP1
atggcccccaaaagaaaaagcggcgtctctaaaagcgagacaaaaagcacaaaggcttgtccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagctgggggattaatttggctacatcagatacagaggattccccaggaaataatacacttcccacatggagtatggcaaagctccagcttcccatgctcaatgaggacctcacgtgtgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 14 - protein, 384 aa, Artificial Sequence
  Description of Artificial SequencePyVP1-R78W (PyVP1-3C-R78W) variant of polyoma virus coat protein VP1
MAPKRKSGVSKSETKSTKACPRPAPVPKLLIKGGMEVLDLVTGPDSVTEIEAFLNPRMGQPPTPESLTEGGQYYGWSWGINLATSDTEDSPGNNTLPTWSMAKLQLPMLNEDLTCDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 15 - DNA, 1263 bp, Artificial Sequence
  Description of Artificial SequencePyVP1-Def assembly-deficient variant of polyoma virus coat protein VP1
atggcccccaaaagaaaaagcggcgtctctaaaagcgagacaaaaagcacaaaggctagcccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacgtctgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggggcagcggcagcggctggacagaacataaatcacctgatggaaggacttattattacaatactgaaacaaaacagtctacctgggaaaagccagatgatggtagtggtagcggcgttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 16 - protein, 421 aa, Artificial Sequence
  Description of Artificial SequencePyVP1-Def assembly-deficient variant of polyoma virus coat protein VP1
MAPKRKSGVSKSETKSTKASPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTSDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWGSGSGWTEHKSPDGRTYYYNTETKQSTWEKPDDGSGSGVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 17 - DNA, 1581 bp, Artificial Sequence
  Description of Artificial Sequencevariant of listeriolysin O (LLO)
atgccgccacctccaccgccacctccgttaccaggcctaggccggcgtgggctagcgacgtccgatgcatctgcattcaataaagaaaatttaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttataaagatggaaatgaatatatcgttgtggagaaaaagaagaaatccatcaatcaaaataatgcagatatccaagttgtgaatgcaatttcgagcctaacatatccaggtgctctcgtgaaagcgaattcggaattagtagaaaatcaacccgatgttcttcctgtcaaacgtgattcattaacacttagcattgatttgccaggaatgactaatcaagacaataaaattgttgtaaaaaatgctactaaatcgaacgttaacaacgcagtaaatacattagtggaaagatggaatgaaaaatatgctcaagcttatccaaatgtaagtgcaaaaattgattatgatgacgaaatggcttacagtgaatcgcaattaattgcaaaatttggtacggcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcatatggccgtcaagtttatttgaaattatcaactaattcccatagtaccaaagtaaaagctgcttttgacgctgccgtaagtgggaaatctgtctcaggtgatgtagaactgacaaatatcatcaaaaattcttccttcaaagccgtaatttacggtggctccgcaaaagatgaagttcaaatcatcgacggtaacctcggagacttacgagatattttgaaaaaaggtgctacttttaaccgggaaacaccaggagttcccattgcctatacaacaaacttcttaaaagacaatgaattagctgttattaaaaacaactcagaatatattgaaacaacttcaaaagcttatacagatggaaaaatcaacatcgatcactctggaggatacgttgctcaattcaacatctcttgggatgaaataaattatgatcctgaaggtaacgaaattgttcaacataaaaactggagcgaaaacaataaaagtaagctagctcatttcacatcgtccatctatttgccaggtaacgcaagaaatattaatgtttacgctaaagaatgcactggtttagcttgggaatggtggagaacggtaattgatgaccggaacctaccgcttgtgaaaaatagaaatatctccatctggggcactacactttatccgaaatatagtaatagtgtagataatccaatcgaacccggg

SEQ ID NO: 18 - protein, 527 aa, Artificial Sequence
  Description of Artificial Sequencevariant of listeriolysin O (LLO)
MPPPPPPPPLPGLGRRGLATSDASAFNKENLISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIEPG

SEQ ID NO: 19 - DNA, 1266 bp, Artificial Sequence
  Description of Artificial SequencePyVP1-WW150 (PyVP1-CallS-WW150) variant of polyoma virus coat protein VP1
atggcccccaaaagaaaaagcggcgtctctaaaagcgagacaaaaagcacaaaggctagcccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacgtctgacaccctacaaatgtgggaggcagtctcagtgaaaaccgaggtggtgggctctggctcactgttagatgtgcatgggttcaacaaacccacagatacaggcagcggcagcggctggacagaacataaatcacctgatggaaggacttattattacaatactgaaacaaaacagtctacctgggaaaagccagatgatggtagtggtagcggcgtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacgtgcaccccacccgtcctgcagttcacaaacaccctgacaactgtgctcctagatgaaaatggagttgggcccctcagcaaaggagaaggtctatacctctcgagcgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 20 - protein, 422 aa, Artificial Sequence
  Description of Artificial SequencePyVP1-WW150 (PyVP1-CallS-WW150) variant of polyoma virus coat protein VP1
MAPKRKSGVSKSETKSTKASPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTSDTLQMWEAVSVKTEVVGSGSLLDVHGFNKPTDTGSGSGWTEHKSPDGRTYYYNTETKQSTWEKPDDGSGSGVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTCTPPVLQFTNTLTTVLLDENGVGPLSKGEGLYLSSVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 21 - DNA, 1152 bp, Artificial Sequence
  Description of Artificial SequencePyVP1-wt variant of polyoma virus coat protein VP1 with modified amino acid positions 383-384
atggcccccaaaagaaaaagcggcgtctctaaatgcgagacaaaatgtacaaaggcctgtccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccaggaaataatacacttcccacatggagtatggcaaagctccagcttcccatgctcaatgaggacctcacctgtgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacaacaactccacccgtcctgcagttcacaaacaccctgacaactgtgctcctagatgaaaatggagttgggcccctctgtaaaggagagggcctatacctctcctgtgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctcccggg

SEQ ID NO: 22 - protein, 384 aa, Artificial Sequence
  Description of Artificial SequencePyVP1-wt variant of polyoma virus coat protein VP1 with modified amino acid positions 383-384
MAPKRKSGVSKCETKCTKACPRPAPVPKLLIKGGMEVLDLVTGPDSVTEIEAFLNPRMGQPPTPESLTEGGQYYGWSRGINLATSDTEDSPGNNTLPTWSMAKLQLPMLNEDLTCDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTTTPPVLQFTNTLTTVLLDENGVGPLCKGEGLYLSCVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPPG

SEQ ID NO: 23 - DNA, 45 bp, Artificial Sequence
  Description of Artificial SequenceC12S, C16S, C20S mutagenesis oligonucleotide
gtctctaaaagcgagacaaaaagcacaaaggctagcccaagaccc

SEQ ID NO: 24 - DNA, 45 bp, Artificial Sequence
  Description of Artificial SequenceC12S, C16S, C20S mutagenesis oligonucleotide
gggtcttgggctagcctttgtgctttttgtctcgcttttagagac

SEQ ID NO: 25 - DNA, 25 bp, Artificial Sequence
  Description of Artificial SequenceC115S mutagenesis oligonucleotide
gaggacctcacgtctgacaccctac

SEQ ID NO: 26 - DNA, 25 bp, Artificial Sequence
  Description of Artificial SequenceC115S mutagenesis oligonucleotide
gtagggtgtcagacgtgaggtcctc

SEQ ID NO: 27 - DNA, 51 bp, Artificial Sequence
  Description of Artificial SequenceC274S, C283S mutagenesis oligonucleotide
gggcccctcagcaaaggagaaggtctatacctctcgagcgtagatataatg

SEQ ID NO: 28 - DNA, 51 bp, Artificial Sequence
  Description of Artificial SequenceC274S, C283S mutagenesis oligonucleotide
cattatatctacgctcgagaggtatagaccttctcctttgctgaggggccc

SEQ ID NO: 29 - DNA, 28 bp, Artificial Sequence
  Description of Artificial SequencePCR oligonucleotide vp1NImp
tatacatatggcccccaaaagaaaaagc

SEQ ID NO: 30 - DNA, 34 bp, Artificial Sequence
  Description of Artificial SequencePCR oligonucleotide vp1CImp
atatcccgggaggaaatacagtctttgtttttcc

SEQ ID NO: 31 - DNA, 33 bp, Artificial Sequence
  Description of Artificial SequencePCR oligonucleotide
atatgaattccagtcattgaagctgccacaagg

SEQ ID NO: 32 - DNA, 29 bp, Artificial Sequence
  Description of Artificial SequenceT249C mutagenesis oligonucleotide
ggacgggtggggtgcacgtgcgtgcagtg

SEQ ID NO: 33 - DNA, 29 bp, Artificial Sequence
  Description of Artificial SequenceT249C mutagenesis oligonucleotide
cactggaggcacgtgcaccccacccgtcc

SEQ ID NO: 34 - DNA, 25 bp, Artificial Sequence
  Description of Artificial SequenceS20C mutagenesis oligonucleotide
gcacaaaggcttgtccaagacccgc

SEQ ID NO: 35 - DNA, 25 bp, Artificial Sequence
  Description of Artificial SequenceS20C mutagenesis oligonucleotide
gcgggtcttggacaagcctttgtgc

SEQ ID NO: 36 - DNA, 79 bp, Artificial Sequence
  Description of Artificial Sequence oligonucleotide for PyVP1-RGD148 insertion of loop segment with flexible Ser-Gly motifs at position 148/149
caacaaacccacagatacagtaaacggcagcggcagcggcagcggcagcggcagtgcaaaaggaatttccactccagtg

SEQ ID NO: 37 - DNA, 82 bp, Artificial Sequence
  Description of Artificial Sequence oligonucleotide for PyVP1-RGD148 insertion of loop segment with flexible Ser-Gly motifs at position 148/149
cactggagtggaaattccttttgcactgccgctgccgctgctgccgctgccgctgccgtttactgtatctgtgggtttgttg

SEQ ID NO: 38 - DNA, 80 bp, Artificial Sequence
  Description of Artificial Sequence oligonucleotide for PyVP1-RGD293 insertion of loop segment with flexible Ser-Gly motifs at position 293/295
gatataatgggctggagagttaccggcagcggcagcggcagcagcggcagcggcagtggctatgatgtccatcactggag

SEQ ID NO: 39 - DNA, 80 bp, Artificial Sequence
  Description of Artificial Sequence oligonucleotide for PyVP1-RGD293 insertion of loop segment with flexible Ser-Gly motifs at position 293/295
ctccagtgatggacatcatagccactgccgctgccgctgctgccgctgccgctgccggtaactctccagcccattatatc

SEQ ID NO: 40 - DNA, 53 bp, Artificial Sequence
  Description of Artificial Sequence oligonucleotide for PyVP1-RGD148 and PyVP1-RGD293 insertion of Arg-Gly-Asp sequence into loop segment with flexible Ser-Gly motifs
cggcagcggcagcggcagcggtcgtggcgatagcggcagcggcagcggcagtg

SEQ ID NO: 41 - DNA, 53 bp, Artificial Sequence
  Description of Artificial Sequence oligonucleotide for PyVP1-RGD148 and PyVP1-RGD293 insertion of Arg-Gly-Asp sequence into loop segment with flexible Ser-Gly motifs
cactgccgctgccgctgccgctatcgccacgaccgctgccgctgccgctgccg

SEQ ID NO: 42 - DNA, 28 bp, Artificial Sequence
  Description of Artificial SequenceR78W mutation oligonucleotide
ctatggttggagctgggggattaatttg

SEQ ID NO: 43 - DNA, 28 bp, Artificial Sequence
  Description of Artificial SequenceR78W mutation oligonucleotide
caaattaatcccccagctccaaccatag

SEQ ID NO: 44 - protein, 38 aa, Artificial Sequence
  Description of Artificial Sequenceassembly-deficient artificial peptide sequence insert
GSGSGWTEHKSPDGRTYYYNTETKQSTWEKPDDGSGSG

SEQ ID NO: 45 - DNA, 39 bp, Artificial Sequence
  Description of Artificial Sequence oligonucleotide for vector pTIP PCR cloning of listeriolysin O (LLO) gene
tatagacgtccgatgcatctgcattcaataaagaaaatt

SEQ ID NO: 46 - DNA, 38 bp, Artificial Sequence
  Description of Artificial Sequence oligonucleotide for vector pTIP PCR cloning of listeriolysin O (LLO) gene
tacttaaggctgcgattggattatctacactattacta

SEQ ID NO: 47 - protein, 10 aa, Artificial Sequence
  Description of Artificial Sequenceproline-rich sequence
PPPPPPPPLP

SEQ ID NO: 48 - DNA, 20 bp, Artificial Sequence
  Description of Artificial Sequence oligonucleotide for listeriolysin O (LLO) gene second PCR cloning from pTIP vector into pET34b vector
gccgccacctccaccgccac

SEQ ID NO: 49 - DNA, 33 bp, Artificial Sequence
  Description of Artificial Sequence oligonucleotide for listeriolysin O (LLO) gene second PCR cloning from pTIP vector into pET34b vector
attagggttcgattggattatctacactattac